(19) United States
(12) Patent Application Publication
(10) Pub. No.: US 2017/0037118 A1
(43) Pub. Date: Feb. 9, 2017

(54) Methods for treating and/or limiting development of diabetes

(71) Applicant: Biocrine AB, Solna (SE)

(72) Inventors: Per-Olof BERGGREN, Solna (SE); Lisa JUNTTI-BERGGREN, Solna (SE)

(21) Appl. No.: 15/299,540

(22) Filed: Oct. 21, 2016

Related U.S. Application Data
(63) Continuation of application No. 14/205,466, filed on Mar. 12, 2014.
(60) Provisional application No. 61/779,508, filed on Mar. 13, 2013.

(51) Int. Cl. C07K 16/18; C12N 15/113; A61K 47/48

(52) U.S. Cl. CPC C07K 16/18; A61K 47/48092; A61K 47/48246; C12N 15/113; C07K 2319/00; C12N 2310/14; C12N 2310/141; C12N 2310/11

(57) ABSTRACT

Provided herein are methods and compositions for limiting development of and/or treating diabetes, involving compounds of formula A-B, wherein A is a pancreatic β cell targeting moiety, and B is an inhibitor of expression and/or activity of Apolipoprotein CIII (apoCIII), protein kinase A (PKA), Src kinase, and/or β1 integrin.

### CROSS REFERENCE

This application is a continuation of U.S. patent application Ser. No. 14/205,466 filed Mar. 12, 2014, which claims priority to U.S. Provisional Patent Application Ser. No. 61/779,508 filed Mar. 13, 2013, each incorporated by reference herein in its entirety.

### BACKGROUND OF THE INVENTION

The International Diabetes Federation (IDF) has estimated that the number of people with diabetes in 2011 was 366 million and this number is expected to grow to 550 million by 2030. The majority of patients are suffering from type-2 diabetes mellitus (T2DM). There is a link between obesity, insulin resistance and the development of T2DM, but the precise underlying mechanisms remain unknown and it becomes paramount to identify potential therapeutic targets that can ultimately address this crucial problem.

Classical target tissues subjective to insulin resistance in T2DM are muscle, liver and fat. These peripheral tissues maintain glucose homeostasis by effectively responding to insulin and failure of insulin to activate its receptors and the downstream signaling cascades result in defect glucose handling. In addition, insulin can stimulate its receptors on the pancreatic β-cell and thereby directly contribute to a positive feedback loop for insulin biosynthesis and secretion.

Apolipoprotein CIII (apoCIII) is a lipoprotein synthesized mainly in the liver, and to a lesser extent in the small intestine. ApoCIII resides on apoB lipoproteins and high density lipoproteins (HDL) and regulates their metabolism by inhibiting lipoprotein lipase (LPL). The expression of apoCIII is increased by insulin deficiency and insulin resistance and it has also been shown that hyperglycemia induces apoCIII transcription. On top of high levels of circulating triglycerides, mice overexpressing apoCIII are more susceptible to high-fat diet induced diabetes. In humans, elevated levels of circulating apoCIII are associated with insulin resistance. Furthermore, patients with type-1 diabetes mellitus (T1DM) were found to have elevated levels of serum apoCIII. This resulted in a higher activity of the voltage-gated Ca²⁺-channels, increased cytoplasmic free Ca²⁺ concentration ([Ca²⁺]ᵢ) and apoptosis, an effect that was blocked by lowering apoCIII.

### SUMMARY OF THE INVENTION

The invention as disclosed herein provides a method for treating or limiting development of diabetes, comprising administering to a subject in need thereof an amount effective of a composition comprising a compound of formula A-B, wherein A is a pancreatic β cell targeting moiety, and B is an inhibitor of expression and/or activity of Apolipoprotein CIII (apoCIII), protein kinase A (PKA), Src kinase, and/or β1 integrin.

In another aspect, a composition of formula A-B is provided, wherein A is a pancreatic β cell targeting moiety, and B is an inhibitor of Apolipoprotein CIII (apoCIII), protein kinase A (PKA), Src kinase, or β1 integrin. In an additional aspect, the composition is of formula A-B-C, wherein C is a compound that permits cell entry of the composition.

In various embodiments, the apoCIII inhibitor is selected from the group consisting of anti-apoCIII antibody, anti-apoCIII aptamer, apoCIII small interfering RNA, apoCIII small internally segmented interfering RNA, apoCIII short hairpin RNA, apoCIII microRNA, and apoCIII antisense oligonucleotides.

In various embodiments, the PKA or Src kinase inhibitor is selected from the group consisting of PP1 analogs, PP2, adenosine 3′,5′-cyclic monophosphorothioate-R, adenosine 3′,5′-cyclic monophosphorothioate H-7, adenosine 3′,5′-cyclic monophosphorothioate H-8, adenosine 3′,5′-cyclic monophosphorothioate H-9, and adenosine 3′,5′-cyclic monophosphorothioate H-89.

In various embodiments, the β1 integrin inhibitor is selected from the group consisting of anti-β1 integrin antibody, anti-β1 integrin aptamer, β1 integrin small interfering RNA, β1 integrin small internally segmented interfering RNA, β1 integrin short hairpin RNA, β1 integrin microRNA, and β1 integrin antisense oligonucleotides.

In various embodiments, the pancreatic β cell specific targeting moiety comprises a moiety that that selectively binds a protein selected from the group consisting of DiGeorge syndrome critical region gene 2 (DGCR2), golgi brefeldin A resistant guanine nucleotide exchange factor 1 (GBF1), orphan G protein-coupled receptor GPR44(GPR44), SerpinB10 (PI-10), FXYD domain containing ion transport regulator 2 (FXYD2), Tetraspanin-7 (TSPAN7), gap junction protein, delta 2, 36 kDa (GJD2), solute carrier family 18 (vesicular monoamine), member 2 (SLC18A2), prokineticin receptor 1 (PROKR1), glutamate receptor, metabotropic 5 (GRM5), neuropeptide Y receptor Y2 (NPY2R), glucagon-like peptide 1 receptor (GLP1R), and transmembrane protein 27 (TMEM27). In further embodiments, the pancreatic β cell specific targeting moiety comprises a moiety selected from the group consisting of glucagon-like peptide-1 (GLP-1), glucagon-like peptide-2 (GLP-2), peptide YY (PYY), neuropeptide Y (NPY), pancreatic peptide (PP), exendin-4, naphthylalanine and naphthylalanine derivatives.

Specific embodiments of the invention will become evident from the following more detailed description of certain embodiments and the claims.

### BRIEF DESCRIPTION OF THE DRAWINGS

FIG. 1 shows exacerbated hyperglycemia and reduced insulin signaling in ob/ob islets across time. (a) Average body weight, (b) non-fasted blood glucose levels and (c) non-fasted plasma insulin levels measured in both ob/ob and age-matched ob/lean mice at 4-, 8- and 12-weeks of age (n=8-11). (d) Pancreatic islet gene expression changes between obese and lean controls at 4-, 8- and 12-weeks of age. mRNA levels were determined by real-time qPCR, normalized to β-actin. Expression levels of ob/ob were compared to those of ob/lean, which were set at 1.0. There was no significant change in ob/lean gene expression over time (n=4-9). Data presented as mean±standard error of the mean (SEM). *P<0.05, ** P<0.01 by student's t-test or ANOVA (Tukey's post-hoc analysis).

FIG. 2 shows changes in insulin signaling cascade in ob/ob islets across time. (a) Western blot analysis of ob/ob islet lysates prepared from animals at 4-, 8- and 12-weeks of age, probed with antibodies specific for phosphorylated-Akt, total Akt, phosphorylated-FoxO1 and γ-tubulin. Bands were quantitated by densitometry. Total FoxO1 immunoblots had poor signal to noise ratios and thus mRNA levels of FoxO1 were measured instead (n=3-4). (b) Pancreatic islet gene expression changes between obese and lean controls at 4-, 8- and 12-weeks of age. mRNA levels were determined by real-time qPCR, normalized to β-actin (n=4-8). (c) Western blot of serum apoCIII with Ponceau S stain as loading control along with the respective densitometry quantitation (n=3). Data presented as mean±SEM. *P<0.05, ** P<0.01 by ANOVA (Tukey's post-hoc analysis).

FIG. 3 shows the relative expression of apoCIII from tissues known to express apoCIII. (a) Liver expression of apoCIII in both ob/lean and ob/ob at age 4, 8 and 12 weeks are compared and contrasted (n=5-7). (b) Small intestine expression of apoCIII in both ob/lean and ob/ob at age 4, 8 and 12 weeks are compared and contrasted (n=5-6). mRNA levels were determined by real-time qPCR, normalized to β-actin. Intron-spanning (exon 3 and 4) real-time qPCR primers used were exactly the same as that used for determining apoCIII expression from pancreatic islet lysates. Data presented as mean±SEM.

FIG. 4 shows expression of apoCIII in rodent and human pancreatic islets. (a) Tissue-specific expression of apoCIII in ob/lean mice. Specific intron-spanning primers were used to determine apoCIII expression by real-time qPCR. Insert shows representative amplification of the entire coding region of apoCIII in the different tissues (n=4-6). (b) ApoCIII expression in sorted rat primary β-cells. Single cells from dissociated rat islets were sorted according to their distinct auto-fluorescence. Cells were subsequently lysed for expression analysis by real-time qPCR. The enrichment of different islet hormones in the sorted cells shows purity of the sorting method (n=4). (c) ApoCIII localization in isolated human islets using an antibody that recognizes the human apoCIII epitope. As controls, apoCIII antibodies were pre-incubated/blocked with recombinant apoCIII prior to use. In the negative control, only the secondary antibody was added (n=3-6). Data presented as mean±SEM.

FIG. 5 shows the relative expression of the different apoCIII isoforms. (a) Illustration showing the difference between the two isoforms of apoCIII as determined by ENSEMBL (transcript ID as shown). Both isoforms have the similar number of exons and the exact same coding sequence (black filled bars). They only differ in the 5′ untranslated region (UTR) corresponding to exon 1 with isoform 2 having a longer 5′UTR (grey filled bar) compared to isoform 1. Isoform 2 also has a shorter 3′UTR in exon 4. Therefore, different intron spanning real-time qPCR primers were employed to determine the relative levels of both isoform 1 and isoform 2 based on the different sequences of exon 1. The different primers shown were tested and shown to have similar priming efficiencies. (b) Relative expression the 2 different apoCIII isoforms in pancreatic islet lysates (n=3). (c) Relative expression of the 2 different apoCIII isoforms in liver lysates (n=3). Data presented as mean±SEM.

FIG. 6 shows overexpression of mouse Myc-apoCIII in Min6 cells and its implications for Ca²⁺ influx. (a) Both media and cells were collected 24 hours post transfection. C-Myc antibody was used for immunoprecipitation and the resulting bound protein was probed with both c-myc and apoCIII antibody. ApoCIII is detected in the apoCIII transfected cell culture media, but neither detected in the lysate nor in the control transfected cell culture media. (b) Mouse apoCIII coding sequence was tagged with a c-myc sequence right after the signal peptide and expressed in Min6 cells. As a control, a ds-tomato construct under the same promoter was used. Representative maximum intensity projection images of c-Myc and C-peptide staining in transfected Min6 cells. (c) Both Myc-apoCIII and control transfected Min6 cells were loaded with Fura-2 to determine [Ca²⁺]ᵢ upon membrane depolarization using 25 mM KCl (n=4). Data presented as mean±SEM. ** P<0.01 by student's t-test.

FIG. 7 shows the effect of apoCIII knockdown in 12-weeks old ob/ob islets. (a) Isolated islets were either directly lysed (ob/ob) or exposed to apoCIII-specific (AS)/inactive (Scr) antisense oligonucleotides for 18 h. ApoCIII expression was measured by real-time qPCR to determine the efficacy of apoCIII knockdown in cultured islets (n=4). (b) Ob/ob and ob/lean islets exposed to either active or inactive apoCIII antisense were loaded with Fura-2 and exposed to 11 mM glucose to determine glucose-stimulated Ca²⁺ influx (n=4-11). (c) 25 mM KCl was used to induce depolarization and subsequent Ca²⁺ influx (n=9-10). (d) Caspase 3/7 activity in ob/ob islets isolated from animals at 4-, 8- and 12-weeks of age (n=4). (e) Decrease in caspase activity in β-cells from 12-weeks old ob/ob mice either incubated overnight with the Ca²⁺-channel blocker verapamil (left column) or exposed to apoCIII antisense (right column) (n=4). Data presented as mean±SEM. *P<0.05, ** P<0.01 by student's t-test or ANOVA (Tukey's post-hoc).

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the present invention provides compositions of formula A-B is provided, wherein A is a pancreatic β cell targeting moiety, and B is an inhibitor of Apolipoprotein CIII (apoCIII), protein kinase A (PKA), Src kinase, or β1 integrin. In an additional aspect, the composition is of formula A-B-C, wherein C is a compound that permits cell entry of the composition.

The inventors have surprisingly discovered that obesity is associated with a local production of apoCIII in pancreatic β-cells. The examples provided herein demonstrate that apoCIII is locally expressed and increased in islets from ob/ob mice that have reduced insulin signaling in their beta cells due to insulin resistance. As these islets contain >90% β-cells, most of the apoCIII expression is derived from the insulin-secreting β-cells. Thus, the compositions of the invention can be used, for example, in treating or limiting development of diabetes.

As used herein, an “inhibitor” of expression and/or activity of ApoCIII, PKA, Src kinase, and/or β1 integrin includes compounds that reduce the transcription of ApoCIII, PKA, Src kinase, and/or β1 integrin DNA into RNA, compounds that reduce translation of the ApoCIII, PKA, Src kinase, and/or β1 integrin RNA into protein, and compounds that reduce the function of ApoCIII, PKA, Src kinase, and/or β1 integrin protein. Such inhibiting can be complete inhibition or partial inhibition, such that the expression and/or activity of the ApoCIII, PKA, Src kinase, and/or β1 integrin is reduced, resulting in a reduced ability to increase intracellular calcium concentration. Such inhibitors are selected from the group consisting of antibodies that bind to ApoCIII, PKA, Src kinase, and/or β1 integrin; aptamers that can interfere with ApoCIII, PKA, Src kinase, and/or β1 integrin activity; antisense oligonucleotides directed against the ApoCIII, PKA, Src kinase, and/or β1 integrin protein, DNA, or mRNA; small interfering RNAs (siRNAs), short hairpin RNAs (shRNAs), microRNAs (miRNA) or small internally segmented interfering RNAs (sisiRNA) directed against the ApoCIII, PKA, Src kinase, and/or β1 integrin protein, DNA, or mRNA, and any other chemical or biological compound that can interfere with ApoCIII, PKA, Src kinase, and/or β1 integrin activity.

In various embodiments, the apoCIII inhibitor is selected from the group consisting of anti-apoCIII antibody, anti-apoCIII aptamer, apoCIII small interfering RNA, apoCIII small internally segmented interfering RNA, apoCIII short hairpin RNA, apoCIII microRNA, and apoCIII antisense oligonucleotides.

In various embodiments, the PKA or Src kinase inhibitor is selected from the group consisting of PP1 analogs, PP2, adenosine 3′,5′-cyclic monophosphorothioate-R, adenosine 3′,5′-cyclic monophosphorothioate H-7, adenosine 3′,5′-cyclic monophosphorothioate H-8, adenosine 3′,5′-cyclic monophosphorothioate H-9, and adenosine 3′,5′-cyclic monophosphorothioate H-89.

In various embodiments, the β1 integrin inhibitor is selected from the group consisting of anti-β1 integrin antibody, anti-β1 integrin aptamer, β1 integrin small interfering RNA, 01 integrin small internally segmented interfering RNA, β1 integrin short hairpin RNA, β1 integrin microRNA, and β1 integrin antisense oligonucleotides.

The pancreatic β cell targeting moieties of the compositions permit targeting of the composition to pancreatic β cells upon administration to a subject in need thereof. In some embodiments the pancreatic β targeting moiety can comprise an antibody, receptor, receptor ligand and the like, which preferentially and/or specifically bind to pancreatic β cells. One or more targeting moieties can be attached to the inhibitor to provide a composition that is capable targeting the composition to pancreatic β cells. In various embodiments, targeting moieties include, but are not limited to peptides that preferentially bind pancreatic β cells, antibodies that bind pancreatic β cells, and receptor ligands that bind pancreatic β cells.

In one embodiment, the pancreatic β cell specific targeting moiety comprises one or more peptides or other moieties that preferentially bind pancreatic β cells, selected from the group consisting of glucagon-like peptide-1 (GLP-1 SEQ ID NO: 73), glucagon-like peptide-2 (GLP-2 SEQ ID NO: 74), peptide YY (PYY SEQ ID NO: 3), neuropeptide Y (NPY SEQ ID NO: 4), pancreatic peptide (PPY SEQ ID NO: 5), exendin-4 (SEQ ID NO: 6), naphthylalanine and naphthylalanine derivatives (sequences shown in Table 1).

In other embodiments, the pancreatic β cell specific targeting moiety comprises a moiety that that selectively binds a pancreatic β cell protein selected from the group consisting of DiGeorge syndrome critical region gene 2 (DGCR2; SEQ ID NO: 7), golgi brefeldin A resistant guanine nucleotide exchange factor 1 (GBF1 SEQ ID NO: 8), orphan G protein-coupled receptor GPR44(GPR44 SEQ ID NO: 9), SerpinB10 (PI-10 SEQ ID NO: 10), FXYD domain containing ion transport regulator 2 (FXYD2 SEQ ID NO: 11), Tetraspanin-7 (TSPAN7 SEQ ID NO: 12), gap junction protein, delta 2, 36 kDa (GJD2 SEQ ID NO: 13), solute carrier family 18 (vesicular monoamine), member 2 (SLC18A2 SEQ ID NO: 14), prokineticin receptor 1 (PROKR1 SEQ ID NO: 15), glutamate receptor, metabotropic 5 (GRM5 SEQ ID NO: 16), neuropeptide Y receptor Y2 (NPY2R SEQ ID NO: 17), glucagon-like peptide 1 receptor (GLP1R SEQ ID NO: 18), and transmembrane protein 27 (TMEM27 SEQ ID NO: 19).

TABLE 1
Amino acid sequences.

| Protein | Sequence |
|---|---|
| DiGeorge syndrome critical | MVPKADSGAELLLELLVLTVTEPLRPELRCNPGQFACRSGTIQCIPLPWQCDG |
| region gene 2 (DGCR2) | WATCEDESDEANCPEVTGEVRPHHGKEAVDPRQGRARGGDPSHFHAVNVAQPV |
| Gene synonyms: | RFSSFLGKCPTGWHHYEGTASCYRVYLSGENYWDAAQTCQRLNGSLATFSTDQ |
| DGS-C; IDD; LAN; SEZ-12 | ELREVLAQEWDQPERSEGWKDQRKLWVGYQYVITGRNRSLEGRWEVAFKGSSE |
| (SEQ ID NO: 7) | VFLPPDPIFASAMSENDNVECAQLQCFHEPTLRHHDLHSWHAESCYEKSSFLC |
| | KRSQTCVDIKDNVVDEGFYFTPKGDDPCLSCTCHGGEPEMCVAALCERPQGCQ |
| | QYRKDPKECCKFMCLDPDGNSLEDSMASGMRLVVSCISSFLILSLLLFMVHRL |
| | RQRRRERIESLIGANLHHENLGRRIPGFDYGPDGEGTGLTPLHLSDDGEGGTF |
| | HEHDPPPPYTAYKYPDIGQPDDPPPPYEASIHPDSVEYDPADDDAFEPVEVSL |
| | PAPGDGGSEGALLRRLEQPLPTAGASLADLEDSADSSSALLVPPDPAQSGSTP |
| | AAEALPGGGRHSRSSLNTVV |
| golgi brefeldin A resistant | MVDKNIYIIQGEINIVVGAIKRNARWSTHTPLDEERDPLLHSFGHLKEVLNSI |
| guanine nucleotide exchange | TELSEIEPNVFLRPFLEVIRSEDTTGPITGLALTSVNKFLSYALIDPTHEGTA |
| factor 1 (GBF1) | EGMENMADAVTHAREVGTDPASDEVVLMKILQVLRTLLLTPVGAHLTNESVCE |
| Gene synonym: | IMQSCFRICFEMRLSELLRKSAEHTLVDMVQLLFTRLPQFKEEPKNYVGTNMK |
| ARF1GEF | KLKMRAGGMSDSSKWKKQKRSPRPPRHMTKVTPGSELPTPNGTTLSSNLTGGM |
| (SEQ ID NO: 8) | PFIDVPTPISSASSEAASAVVSPSTDSGLEFSSQTTSKEDLTDLEQPGSPGYS |
| | TATEPGSSELGVPEQPDLQEGTHVEKSQSASVESIPEVLEECTSPADHSDSAS |
| | VHDMDYVNPRGVRFTQSSQKEGTALVPYGLPCIRELFRFLISLTNPHDRHNSE |
| | VMIHMGLHLLTVALESAPVAQCQTLLGLIKDEMCRHLFQLLSIERLNLYAASL |
| | RVCELLFESMREHLKFQMEMYIKKLMETITVENPKMPYEMKEMALEAIVQLWR |
| | IPSFVTELYINYDCDYYCSNLFEELTKLLSKNAFPVSGQLYTTHLLSLDALLT |
| | VIDSTEAHCQAKVLNSLTQQEKKETARPSCEIVDGTREASNTERTASDGKAVG |
| | MASDIPGLHLPGGGRLPPEHGKSGCSDLEEAVDSGADKKFARKPPRESCLLPD |
| | PRELIEIKNKKKLLITGTEQFNQKPKKGIQFLQEKGLLTIPMDNTEVAQWLRE |
| | NPRLDKKMIGEFVSDRKNIDLLESEVSTFSFQGLRLDEALRLYLEAFRLPGEA |
| | PVIQRLLEAFTERWMNCNGSPFANSDACFSLAYAVIMLNTDQHNHNVRKQNAP |
| | MTLEEFRKNLKGVNGGKDFEQDILEDMYHAIKNEEIVMPEEQTGLVRENYVWN |
| | VLLHRGATPEGIFLRVPTASYDLDLFTMTWGPTIAALSYVEDKSLEETTIQKA |
| | ISGERKCAMISAHYGLSDVEDNLIISLCKFTALSSESIENLPSVEGSNPKAHI |
| | AAKTVEHLAHRHGDILREGWKNIMEAMLQLFRAQLLPKAMIEVEDEVDPNGKI |
| | SLQREETPSNRGESTVLSEVSWLTLSGPEQSSVRGPSTENQEAKRVALECIKQ |
| | CDPEKMITESKFLQLESLQELMKALVSVTPDEETYDEEDAAFCLEMLLRIVLE |
| | NRDRVGCVWQTVRDHLYHLCVQAQDFCELVERAVVGLLRLAIRLLRREEISAQ |
| | VLLSLRILLLMKPSVLSRVSHQVAYGLHELLKTNAANIHSGDDWATLFTLLEC |
| | IGSGVKPPAALQATARADAPDAGAQSDSELPSYHQNDVSLDRGYTSDSEVYTD |
| | HGRPGKIHRSATDADVVNSGWLVVGKDDVDNSKPGPSRPGPSPLINQYSLTVG |
| | LDLGPHDTKSLLKCVESLSFIVRDAAHITPDNFELCVKTLRIFVEASLNGGCK |
| | SQEKRGKSHKYDSKGNRFKKKSKEGSMLRRPRTSSQHASRGGQSDDDEDEGVP |
| | ASYHTVSLQVSQDLLDLMHTLHTRAASIYSSWAEEQRHLETGGQKIEADSRTL |
| | WAHCWCPLLQGIACLCCDARRQVRMQALTYLQRALLVHDLQKLDALEWESCFN |
| | KVLFPLLTKLLENISPADVGGMEETRMRASTLLSKVFLQHLSPLLSLSTFAAL |
| | WLTILDFMDKYMHAGSSDLLSEAIPESLKNMLLVMDTAEIFHSADARGGGPSA |
| | LWEITWERIDCFLPHLRDELFKQTVIQDPMPMEPQGQKPLASAHLTSAAGDIR |
| | TPGHPPPPEIPSELGACDFEKPESPRAASSSSPGSPVASSPSRLSPTPDGPPP |
| | LAQPPLILQPLASPLQVGVPPMTLPIILNPALIEATSPVPLLATPRPTDPIPT |
| | SEVN |
| orphan G protein-coupled | MTDIQVSEAEKGSGRLNDLPKHSNAGILFPPPPPPNIFVGHIGISWAGSHSGP |
| receptor GPR44 (GPR44) | LSWFPGGLCTSNLGASEPPLQSPTMSANATLKPLCPILEQMSRLQSHSNTSIR |
| Gene synonym: | YIDHAAVLLHGLASLLGLVENGVILFVVGCRMRQTVVTTWVLHLALSDLLASA |
| GPR44 | SLPFETYFLAVGHSWELGTTECKLHSSIFELNMFASGELLSAISLDRCLQVVR |
| (SEQ ID NO: 9) | PVWAQNHRTVAAAHKVCLVLWALAVLNTVPYFVERDTISRLDGRIMCYYNVLL |
| | LNPGPDRDATCNSRQAALAVSKELLAFLVPLAIIASSHAAVSLRLQHRGRRRP |
| | GRFVRLVAAVVAAFALCWGPYHVFSLLEARAHANPGLRPLVWRGLPFVTSLAF |
| | FNSVANPVLYVLTCPDMLRKLRRSLRTVLESVLVDDSELGGAGSSRRRRTSST |
| | ARSASPLALCSRPEEPRGPARLLGWLLVQLRSVPADGPPEPGAEQHLELEPGP |
| | RRAALTRESITRVPRFNSISGLLPQ |
| SerpinB10 (PI-10) | MDSLATSINQFALELSKKLAESAQGKNIFFSSWSISTSLTIVYLGAKGTTAAQ |
| Gene synonyms: | MAQVLQFNRDQGVKCDPESEKKRKMEFNLSNSEEIHSDFQTLISEILKPNDDY |
| PI-10; PI10 | LLKTANAIYGEKTYAFHNKYLEDMKTYFGAEPQPVNEVEASDQIRKDINSWVE |
| (SEQ ID NO: 10) | RQTEGKIQNLLPDDSVDSTIRMILVNALYFKGIWEHQFLVQNTTEKPFRINET |
| | TSKPVQMMFMKKKLHIFHIEKPKAVGLQLYYKSRDLSLLILLPEDINGLEQLE |
| | KAITYEKLNEWTSADMMELYEVQLHLPKFKLEDSYDLKSTLSSMGMSDAFSQS |
| | KADFSGMSSARNLELSNVEHKAFVEINEQGTEAAAGSGSEIDIRIRVPSIEFN |
| | ANHPFLFFIRHNKTNTILFYGRLCSP |
| FXYD domain containing ion | Isoform 1: |
| transport regulator 2 (FXYD2) | MTGLSMDGGGSPKGDVDPFYYDYETVRNGGLIFAGLAFIVGLLILLSRRFRCG |
| Gene synonyms: | GNKKRRQINEDEP |
| ATP1G1; HOMG2 | Isoform 2: (SEQ ID NO: 72) |
| (SEQ ID NO: 11) | MDRWYLGGSPKGDVDPFYYDYETVRNGGLIFAGLAFIVGLLILLSRRFRCGGN |
| | KKRRQINEDEP |
| Tetraspanin-7 (TSPAN7) | MASRRMETKPVITCLKTLLIIYSFVFWITGVILLAVGVWGKLTLGTYISLIAE |
| Gene synonyms: | NSTNAPYVLIGTGITIVVEGLEGCFATCRGSPWMLKLYAMFLSLVFLAELVAG |
| A15; CCG-B7; CD231; | ISGFVERHEIKDTFLRTYTDAMQTYNGNDERSRAVDHVQRSLSCCGVQNYTNW |
| DXS1692E; MRX58; MXS1; | STSPYFLEHGIPPSCCMNETDCNPQDLHNLIVAATKVNQKGCYDLVISFMETN |
| TALLA-1; TM4SF2; | MGIIAGVAFGIAFSQLIGMLLACCLSRFITANQYEMV |
| TM4SF2b | |
| (SEQ ID NO: 12) | |
| gap junction protein, delta 2, | MGEWTILERLLEAAVQQHSTMIGRILLTVVVIFRILIVAIVGETVYDDEQTMF |
| 36 kDa (GJD2) | VCNTLQPGCNQACYDRAFPISHIRYWVFQIIMVCIPSLCFITYSVHQSAKQRE |
| Gene synonyms: | RRYSTVFLALDRDPPESIGGPGGIGGGGSGGGKREDKKLQNAIVNGVLQNTEN |
| CX36; GJA9 | TSKETEPDCLEVKELTPHPSGLRTASKSKLRRQEGISRFYIIQVVFRNALEIG |
| (SEQ ID NO: 13) | FLVGQYFLYGESVPGLYECNRYPCIKEVECYVSRPTEKTVELVFMFAVSGICV |
| | VLNLAELNHLGWRKIKLAVRGAQAKRKSIYEIRNKDLPRVSVPNEGRTQSSDS |
| | AYV |
| solute carrier family 18 | MALSELALVRWLQESRRSRKLILFIVFLALLLDNMLLTVVVPIIPSYLYSIKH |
| (vesicular monoamine), | EKNATEIQTARPVHTASISDSFQSIFSYYDNSTMVIGNATRDLTLHQTATQHM |
| member 2 (SLC18A2) | VINASAVPSDCPSEDKDLLNENVQVGLLFASKATVQLITNPFIGLLTNRIGYP |
| Gene synonyms: | IPIFAGFCIMFVSTIMFAFSSSYAFLLIARSLQGIGSSCSSVAGMGMLASVYT |
| SVAT; SVMT; VAT2; | DDEERGNVMGIALGGLAMGVLVGPPFGSVLYEFVGKTAPFLVLAALVLLDGAI |
| VMAT2 | QLFVLQPSRVQPESQKGTPLTTLLKDPYILIAAGSICFANMGIAMLEPALPIW |
| (SEQ ID NO: 14) | MMETMCSRKWQLGVAFLPASISYLIGTNIFGILAHKMGRWLCALLGMIIVGVS |
| | ILCIPFAKNIYGLIAPNFGVGFAIGMVDSSMMPIMGYLVDLRHVSVYGSVYAI |
| | ADVAFCMGYAIGPSAGGAIAKAIGFPWLMTIIGilDILFAPLCFFLRSPPAKE |
| | EKMAILMDHNCPIKTKMYTQNNIQSYPIGEDEESESD |
| prokineticin receptor 1 | METTMGFMDDNATNTSTSFLSVLNPHGAHATSFPFNFSYSDYDMPLDEDEDVT |
| (PROKR1) | NSRTFFAAKIVIGMALVGIMLVCGIGNFIFIAALVRYKKLRNLTNLLIANLAI |
| Gene synonyms: | SDFLVAIVCCPFEMDYYVVRQLSWEHGHVLCTSVNYLRTVSLYVSTNALLAIA |
| GPR73; GPR73a; PKR1; ZAQ | IDRYLAIVHPLRPRMKCQTATGLIALVWTVSILIAIPSAYFTTETVLVIVKSQ |
| (SEQ ID NO: 15) | EKIFCGQIWPVDQQLYYKSYFLFIFGIEFVGPVVTMTLCYARISRELWFKAVP |
| | GFQTEQIRKRLRCRRKTVLVLMCILTAYVLCWAPFYGFTIVRDFFPTVFVKEK |
| | HYLTAFYIVECIAMSNSMINTLCFVTVKNDTVKYFKKIMLLHWKASYNGGKSS |
| | ADLDLKTIGMPATEEVDCIRLK |
| glutamate receptor, | MVLLLILSVLLLKEDVRGSAQSSERRVVAHMPGDIIIGALFSVHHQPTVDKVH |
| metabotropic 5 (GRM5) | ERKCGAVREQYGIQRVEAMLHTLERINSDPTLLPNITLGCEIRDSCWHSAVAL |
| Gene synonyms: | EQSIEFIRDSLISSEEEEGLVRCVDGSSSSFRSKKPIVGVIGPGSSSVAIQVQ |
| GPRC1E; mGlu5; MGLUR5 | NLLQLFNIPQIAYSATSMDLSDKTLFKYFMRVVPSDAQQARAMVDIVKRYNWT |
| (SEQ ID NO: 16) | YVSAVHTEGNYGESGMEAFKDMSAKEGICIAHSYKIYSNAGEQSFDKLLKKLT |
| | SHLPKARVVACFCEGMTVRGLLMAMRRLGLAGEFLLLGSDGWADRYDVTDGYQ |
| | REAVGGITIKLQSPDVKWFDDYYLKLRPETNHRNPWFQEFWQHRFQCRLEGFP |
| | QENSKYNKTCNSSLTLKTHHVQDSKMGFVINAIYSMAYGLHNMQMSLCPGYAG |
| | LCDAMKPIDGRKLLESLMKTNFTGVSGDTILFDENGDSPGRYEIMNFKEMGKD |
| | YFDYINVGSWDNGELKMDDDEVWSKKSNIIRSVCSEPCEKGQIKVIRKGEVSC |
| | CWTCTPCKENEYVFDEYTCKACQLGSWPTDDLTGCDLIPVQYLRWGDPEPIAA |
| | VVFACLGLLATLFVTVVFIIYRDTPVVKSSSRELCYIILAGICLGYLCTFCLI |
| | AKPKQIYCYLQRIGIGLSPAMSYSALVTKTNRIARILAGSKKKICTKKPRFMS |
| | ACAQLVIAFILICIQLGIIVALFIMEPPDIMHDYPSIREVYLICNTTNLGVVT |
| | PLGYNGLLILSCTFYAFKTRNVPANFNEAKYIAFTMYTTCIIWLAFVPIYFGS |
| | NYKIITMCFSVSLSATVALGCMFVPKVYIILAKPERNVRSAFTTSTVVRMHVG |
| | DGKSSSAASRSSSLVNLWKRRGSSGETLRYKDRRLAQHKSEIECFTPKGSMGN |
| | GGRATMSSSNGKSVTWAQNEKSSRGQHLWQRLSIHINKKENPNQTAVIKPFPK |
| | STESRGLGAGAGAGGSAGGVGATGGAGCAGAGPGGPESPDAGPKALYDVAEAE |
| | EHFPAPARPRSPSPISTLSHRAGSASRTDDDVPSLHSEPVARSSSSQGSLMEQ |
| | ISSVVTRFTANISELNSMMLSTAAPSPGVGAPLCSSYLIPKEIQLPTTMTTFA |
| | EIQPLPAIEVTGGAQPAAGAQAAGDAARESPAAGPEAAAAKPDLEELVALTPP |
| | SPFRDSVDSGSTTPNSPVSESALCIPSSPKYDTLIIRDYTQSSSSL |
| neuropeptide Y receptor Y2 | MGPIGAEADENQTVEEMKVEQYGPQTTPRGELVPDPEPELIDSTKLIEVQVVL |
| (NPY2R) | ILAYCSIILLGVIGNSLVIHVVIKFKSMRTVTNFFIANLAVADLLVNTLCLPF |
| Gene synonym: | TLTYTLMGEWKMGPVLCHLVPYAQGLAVQVSTITLTVIALDRHRCIVYHLESK |
| NPY2-R | ISKRISFLIIGLAWGISALLASPLAIFREYSLIEIIPDFEIVACTEKWPGEEK |
| (SEQ ID NO: 17) | SIYGTVYSLSSLLILYVLPLGIISFSYTRIWSKLKNHVSPGAANDHYHQRRQK |
| | TTKMLVCVVVVFAVSWLPLHAFQLAVDIDSQVLDLKEYKLIFTVFHIIAMCST |
| | FANPLLYGWMNSNYRKAFLSAFRCEQRLDAIHSEVSVTFKAKKNLEVRKNSGP |
| | NDSFTEATNV |
| glucagon-like peptide 1 | MAGAPGPLRLALLLLGMVGRAGPRPQGATVSLWETVQKWREYRRQCQRSLTED |
| receptor (GLP1R) | PPPATDLFCNRTFDEYACWPDGEPGSFVNVSCPWYLPWASSVPQGHVYRFCTA |
| Gene synonyms: | EGLWLQKDNSSLPWRDLSECEESKRGERSSPEEQLLFLYIIYTVGYALSFSAL |
| GLP-1R; GLP-1-R; GLP-1 | VIASAILLGFRHLHCTRNYIHLNLFASFILRALSVFIKDAALKWMYSTAAQQH |
| receptor | QWDGLLSYQDSLSCRLVFLLMQYCVAANYYWLLVEGVYLYTLLAFSVLSEQWI |
| (SEQ ID NO: 18) | FRLYVSIGWGVPLLFVVPWGIVKYLYEDEGCWTRNSNMNYWLIIRLPILFAIG |
| | VNFLIFVRVICIVVSKLKANLMCKTDIKCRLAKSTLTLIPLLGTHEVIFAFVM |
| | DEHARGTLRFIKLFTELSFTSFQGLMVAILYCFVNNEVQLEFRKSWERWRLEH |
| | LHIQRDSSMKPLKCPTSSLSSGATAGSSMYTATCQASCS |
| transmembrane protein 27 | MLWLLFFLVTAIHAELCQPGAENAFKVRLSIRTALGDKAYAWDTNEEYLFKAM |
| (TMEM27) | VAFSMRKVPNREATEISHVLLCNVTQRVSFWFVVTDPSKNHTLPAVEVQSAIR |
| Gene synonyms: | MNKNRINNAFFLNDQTLEFLKIPSTLAPPMDPSVPIWIIIFGVIFCIIIVAIA |
| NX-17; NX17 | LLILSGIWQRRRKNKEPSEVDDAEDKCENMITIENGIPSDPLDMKGGHINDAF |
| (SEQ ID NO: 19) | MTEDERLTPL |
| glucagon (GCG) | MKSIYFVAGLFVMLVQGSWQRSLQDTEEKSRSFSASQADPLSDPDQMNEDKRH |
| Gene synonyms: | SQGTFTSDYSKYLDSRRAQDFVQWLMNIKRNRNNIAKRHDEFERHAEGIFTSD |
| GLP1; GLP2; GRPP | VSSYLEGQAAKEFIAWLVKGRGRRDFPEEVAIVEELGRRHADGSFSDEMNTIL |
| (SEQ ID NO: 1) | DNLAARDFINWLIQTKITDRK |
| | Glucagon-like peptide 1; GLP1(aa92-128) |
| | (SEQ ID NO: 73) |
| | HDEFERHAEGTFTSDVSSYLEGQAAKEFIAWLVKGRG |
| | Glucagon-like peptide 2; GLP2(aa146-178) |
| | (SEQ ID NO: 74) |
| | HADGSFSDEMNTILDNLAARDFINWLIQTKITD |
| pancreatic peptide (PPY) | MAAARLCLSLLLLSTCVALLLQPLLGAQGAPLEPVYPGDNATPEQMAQYAADL |
| Gene synonyms: | RRYINMLTRPRYGKRHKEDTLAFSEWGSPHAAVPRELSPLDL |
| PNP; PP | |
| (SEQ ID NO: 5) | |
| neuropeptide Y (NPY) | MLGNKRLGLSGLTLALSLLVCLGALAEAYPSKPDNPGEDAPAEDMARYYSALR |
| Gene synonym: | HYINLITRQRYGKRSSPETLISDLLMRESTENVPRTRLEDPAMW |
| PYY4 | |
| (SEQ ID NO: 4) | |
| peptide YY (PYY) | MVFVRRPWPALTIVLLALLVCLGALVDAYPIKPEAPGEDASPEELNRYYASLR |
| Gene synonyms: | HYLNLVTRQRYGKRDGPDTLLSKTFFPDGEDRPVRSRSEGPDLW |
| PYY-I; PYY1 | |
| (SEQ ID NO: 3) | |
| exendin-4 | MKIILWLCVFGLFLATLFPISWQMPVESGLSSEDSASSESFASKIKRHGEGIF |
| Gene synonym: | TSDLSKQMEEEAVRLFIEWLKNGGPSSGAPPPSG |
| (SEQ ID NO: 6) | |
| apolipoprotein C-III (APOC3) | APOC3 Protein; SEQ ID NO.: 75 |
| Homo sapiens | MQPRVLLVVALLALLASARASEAEDASLLSFMQGYMKHATKTAKDALSSVQES |
| Gene synonyms: | QVAQQARGWVTDGFSSLKDYWSTVKDKFSEFWDLDPEVRPTSAVAA |
| HALP2; APOCIII | APOC3 Gene; SEQ ID NO.: 76 |
| | tgctcagttc atccctagag gcagctgctc caggaacaga |
| | ggtgccatgc agccccgggt actccttgtt gttgccctcc |
| | tggcgctcct ggcctctgcc cgagcttcag aggccgagga |
| | tgcctccctt ctcagcttca tgcagggtta catgaagcac |
| | gccaccaaga ccgccaagga tgcactgagc agcgtgcagg |
| | agtcccaggt ggcccagcag gccaggggct gggtgaccga |
| | tggcttcagt tccctgaaag actactggag caccgttaag |
| | gacaagttct ctgagttctg ggatttggac cctgaggtca |
| | gaccaacttc agccgtggct gcctgagacc tcaatacccc |
| | aagtccacct gcctatccat cctgcgagct ccttgggtcc |
| | tgcaatctcc agggctgccc ctgtaggttg cttaaaaggg |
| | acagtattct cagtgctctc ctaccccacc tcatgcctgg |
| | cccccctcca ggcatgctgg cctcccaata aagctggaca |
| | agaagctgct atg |

When the targeting moiety comprises an antibody, such antibodies can be polyclonal or monoclonal. The antibodies can be humanized, fully human, or murine forms of the antibodies. Such antibodies can be made by well-known methods, such as described in Harlow and Lane, Antibodies; A Laboratory Manual, Cold Spring Harbor Laboratory, Cold Spring Harbor, N.Y., (1988). In some embodiments, additional amino acid residues may be added to either the N- or C-terminus of the antibody or antibody fragment. When the targeting moiety comprises an aptamer, such aptamers can be oligonucleic acid or peptide molecules that bind to a specific target molecule. Methods of constructing and determining the binding characteristics of aptamers are well known in the art, and the aptamers can be isolated from random libraries or they can be previously identified peptides.

Attaching the pancreatic β cell targeting moiety to the inhibitor may be accomplished by any chemical reaction that will bind the two molecules so long as the pancreatic β cell targeting moiety and the inhibitor retain their respective activities. In one embodiment, both the β cell targeting moiety and the inhibitor comprise polypeptides, and the composition comprises a recombinant fusion protein. In another embodiment, both the β cell targeting moiety and the inhibitor comprise polynucleotides, and the composition comprises a recombinant nucleic acid. In other embodiments, a linkage between the pancreatic β cell targeting moiety and the inhibitor can include many chemical mechanisms, for instance covalent binding, affinity binding, intercalation, coordinate binding and complexation. Covalent binding can be achieved either by direct condensation of existing side chains or by the incorporation of external bridging molecules. Many bivalent or polyvalent linking agents are useful in coupling protein molecules, such as the antibodies, to other molecules. For example, representative, non-limiting examples of coupling agents can be organic compounds such as thioesters, carbodiimides, succinimide esters, disocyanates, glutaraldehydes, diazobenzenes and hexamethylene diamines.

In another embodiment, the compositions are of general formula A-B-C, wherein C is a compound that permits cell entry of the composition. In one embodiment, the compound that permits cell entry of the composition may comprise a cell penetrating peptide. In other embodiments, the compound that permits cell entry of the composition may comprise a non-peptidic cell penetrating compound. Cell penetrating peptides or non-peptidic cell penetrating compounds facilitate uptake of the composition into the pancreatic β cells targeted by the targeting moiety of the composition. The cell penetrating compound can be linked to components A-B of the composition by any suitable technique that retains the activities of each component, as discussed herein. In one embodiment, each of A-B-C are polypeptides and the composition comprises a fusion protein.

Exemplary cell penetrating peptides include, but are not limited to RRASAP (SEQ ID NO: 20) LRRASAP (SEQ ID NO: 21) WLRRASAP (SEQ ID NO: 22) RRATAP (SEQ ID NO: 23) LRRATAP (SEQ ID NO: 24) WLRRATAP (SEQ ID NO: 25) RRAYAP (SEQ ID NO: 26) LRRAYAP (SEQ ID NO: 27) WLRRAYAP (SEQ ID NO: 28) RRADAP (SEQ ID NO: 29) LRRADAP (SEQ ID NO: 30) WLRRADAP (SEQ ID NO: 31) RRAEAP (SEQ ID NO: 32) LRRAEAP (SEQ ID NO: 33) WLRRAEAP (SEQ ID NO: 34) RRASAPRRASAP (SEQ ID NO: 35) LRRASAPLRRASAP (SEQ ID NO: 36) WLRRASAPWLRRASAP (SEQ ID NO: 37) RRATAPRRATAP (SEQ ID NO: 38) LRRATAPLRRATAP (SEQ ID NO: 39) WLRRATAPWLRRATAP (SEQ ID NO: 40) RRAYAPRRAYAP (SEQ ID NO: 41) LRRAYAPLRRAYAP (SEQ ID NO 42) WLRRAYAPWLRRAYAP (SEQ ID NO: 43) RRADAPRRADAP (SEQ ID NO: 44) LRRADAPLRRADAP (SEQ ID NO: 45) WLRRADAPWLRRADAP (SEQ ID NO: 46) RRAEAPRRAEAP (SEQ ID NO: 47) LRRAEAPLRRAEAP (SEQ ID NO: 48) WLRRAEAPWLRRAEAP (SEQ ID NO: 49), GRKKRRQRRRPPQ (SEQ ID NO:50); AYARAAARQARA (SEQ ID NO:51); DAATATRGRSAASRPTERPRAPARSASRPRRPVE (SEQ ID NO:52); GWTLNSAGYLLGLINLKALAALAKKIL (SEQ ID NO:53); PLSSISRIGDP (SEQ ID NO:54); AAVALLPAVLLALLAP (SEQ ID NO:55); AAVLLPVLLAAP (SEQ ID NO:56); VTVLALGALAGVGVG (SEQ ID NO:57); GALFLGWLGAAGSTMGAWSQP (SEQ ID NO:58); GWTLNSAGYLLGLINLKALAALAKKIL (SEQ ID NO:59); KLALKLALKALKAALKLA (SEQ ID NO:60); KETWWETWWTEWSQPKKKRKV (SEQ ID NO:61); KAFAKLAARLYRKAGC (SEQ ID NO:62); KAFAKLAARLYRAAGC (SEQ ID NO:63); AAFAKLAARLYRKAGC (SEQ ID NO:64); KAFAALAARLYRKAGC (SEQ ID NO:65); KAFAKLAAQLYRKAGC (SEQ ID NO:66), AGGGGYGRKKRRQRRR (SEQ ID NO:67); YGRKKRRQRRR (SEQ ID NO:68); and YARAAARQARA (SEQ ID 69).

The individual components of the composition can be made by any suitable means known to those of skill in the art. Once the composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In a second aspect, the present invention provides an isolated nucleic acid encoding a fusion protein or recombinant nucleic acid of any embodiment or combination of embodiments of the compositions of the invention, operatively linked to a promoter. Thus, in some embodiments the fusion protein comprises a fusion protein of any embodiment or combination of embodiments of peptidic components A-B (and optionally C) of the compositions of the invention. In other embodiments, components A-B of the compositions of the invention are each polynucleotides, and thus the isolated nucleic acid encodes a recombinant nucleic acid composition of the invention. The isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. The term “operatively linked” is used herein to refer to an arrangement of flanking sequences wherein the flanking sequences so described are configured or assembled so as to perform their usual function. Thus, a flanking sequence operably linked to a coding sequence may be capable of effecting the replication, transcription, and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

In a third aspect, the present invention provides recombinant expression vectors comprising an isolated nucleic acid of the invention. The term “vector” as used herein refers to any molecule (e.g., nucleic acid, plasmid, or virus) that is used to transfer coding information to a host cell. The term “vector” includes a nucleic acid molecule that is capable of transporting another nucleic acid to which it has been linked. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms “plasmid” and “vector” may be used interchangeably herein, as a plasmid is the most commonly used form of vector. However, the invention is intended to include other forms of expression vectors, such as viral vectors (e.g., replication defective retroviruses, adenoviruses, and adeno-associated viruses), which serve equivalent functions.

In a fourth aspect, the present invention provides recombinant host cells comprising an expression vector of any embodiment or combination of embodiments of the invention. The phrase “recombinant host cell” (or “host cell”) as used herein refers to a cell into which a recombinant expression vector has been introduced. A recombinant host cell or host cell is intended to refer not only to the particular subject cell, but also to the progeny of such a cell. Because certain modifications may occur in succeeding generations due to either mutation or environmental influences, such progeny may not, in fact, be identical to the parent cell, but such cells are still included within the scope of the term “host cell” as used herein. A wide variety of host cell expression systems can be used to express the antibody-like binding proteins of the invention, including bacterial, yeast, baculoviral, and mammalian expression systems (as well as phage display expression systems). An example of a suitable bacterial expression vector is pUC19. To express an antibody-like binding protein recombinantly, a host cell is transformed or transfected with one or more recombinant expression vectors carrying DNA fragments encoding the polypeptide chains of the antibody-like binding protein such that the polypeptide chains are expressed in the host cell and, preferably, secreted into the medium in which the host cells are cultured, from which medium the antibody-like binding protein can be recovered.

The term “transformation” as used herein refers to a change in a cell's genetic characteristics, and a cell has been transformed when it has been modified to contain a new DNA. For example, a cell is transformed where it is genetically modified from its native state. Following transformation, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “transfection” as used herein refers to the uptake of foreign or exogenous DNA by a cell, and a cell has been “transfected” when the exogenous DNA has been introduced inside the cell membrane. A number of transfection techniques are well known in the art. Such techniques can be used to introduce one or more exogenous DNA molecules into suitable host cells.

In a fifth aspect, the invention provides methods for treating or limiting development of diabetes, comprising administering to a subject in need thereof an amount effective of a composition comprising a compound of formula A-B, wherein A is a pancreatic β cell targeting moiety, and B is an inhibitor of expression and/or activity of Apolipoprotein CIII (apoCIII), protein kinase A (PKA), Src kinase, and/or β1 integrin. Thus, any embodiment or combination of embodiments of the compositions disclosed herein may be used in the present invention.

In various embodiments, the apoCIII inhibitor is selected from the group consisting of anti-apoCIII antibody, anti-apoCIII aptamer, apoCIII small interfering RNA, apoCIII small internally segmented interfering RNA, apoCIII short hairpin RNA, apoCIII microRNA, and apoCIII antisense oligonucleotides.

In various embodiments, the PKA or Src kinase inhibitor is selected from the group consisting of PP1 analogs, PP2, adenosine 3′,5′-cyclic monophosphorothioate-R, adenosine 3′,5′-cyclic monophosphorothioate H-7, adenosine 3′,5′-cyclic monophosphorothioate H-8, adenosine 3′,5′-cyclic monophosphorothioate H-9, and adenosine 3′,5′-cyclic monophosphorothioate H-89.

In various embodiments, the β1 integrin inhibitor is selected from the group consisting of anti-β1 integrin antibody, anti-β1 integrin aptamer, β1 integrin small interfering RNA, β1 integrin small internally segmented interfering RNA, β1 integrin short hairpin RNA, β1 integrin microRNA, and β1 integrin antisense oligonucleotides.

In various embodiments, the pancreatic β cell specific targeting moiety comprises a moiety that that selectively binds a protein selected from the group consisting of DiGeorge syndrome critical region gene 2 (DGCR2), golgi brefeldin A resistant guanine nucleotide exchange factor 1 (GBF1), orphan G protein-coupled receptor GPR44(GPR44), SerpinB10 (PI-10), FXYD domain containing ion transport regulator 2 (FXYD2), Tetraspanin-7 (TSPAN7), gap junction protein, delta 2, 36 kDa (GJD2), solute carrier family 18 (vesicular monoamine), member 2 (SLC18A2), prokineticin receptor 1 (PROKR1), glutamate receptor, metabotropic 5 (GRM5), neuropeptide Y receptor Y2 (NPY2R), glucagon-like peptide 1 receptor (GLP1R), and transmembrane protein 27 (TMEM27). In further embodiments, the pancreatic β cell specific targeting moiety comprises a moiety selected from the group consisting of glucagon-like peptide-1 (GLP-1), glucagon-like peptide-2 (GLP-2), peptide YY (PYY), neuropeptide Y (NPY), pancreatic peptide (PP), exendin-4, naphthylalanine and naphthylalanine derivatives.

As used herein, “pancreatic β cells” are any population of cells that contains pancreatic β islet cells. Such pancreatic β islet cell populations include the pancreas, isolated pancreatic islets of Langerhans (“pancreatic islets”) and isolated pancreatic β islet cells.

In one embodiment, the method is for treating diabetes. In this embodiment, the subject has been diagnosed with type 1 or type 2 diabetes. As used herein, “diabetes” is characterized by insufficient or no production of insulin by the pancreas, leading to high blood sugar levels.

As used herein, “treating diabetes” means accomplishing one or more of the following: (a) reducing the severity of the diabetes or diabetic complications; (b) limiting or preventing development of diabetic complications; (c) inhibiting worsening of diabetic complications or of symptoms characteristic of diabetes; (d) limiting or preventing recurrence diabetic complications or of symptoms characteristic of diabetes; (e) limiting or preventing recurrence of diabetic complications or of symptoms characteristic of diabetes in patients that were previously symptomatic.

Symptoms characteristic of diabetes that can be treated by the methods of the invention include, but are not limited to, elevated blood glucose levels, decreased insulin production, insulin resistance, proteinuria, and impaired glomerular clearance. Diabetic complications that can be treated according to the methods of the invention include, but are not limited to, complications in the nerves (such as diabetic neuropathy) and complications associated with smooth muscle cell dysregulaton (including but not limited to erectile dysfunction, bladder dysfunction, and vascular complications including but not limited to atherosclerosis, stroke, and peripheral vascular disease)

In another embodiment, the method is for limiting development of diabetes. In this aspect, the subject is at risk of type 1 or type 2 diabetes, and a benefit is to limit development of diabetes and/or diabetic complications. Any subject at risk of developing diabetes can be treated, including but not limited to subjects with one or more of, metabolic syndrome, known genetic risk factors for diabetes, a family history of diabetes, and obesity.

In a further embodiment, the methods for treating or limiting development of diabetes and/or diabetic complications further comprises treating those individuals that have been identified as overexpressing apoCIII compared to control. Increases in apoCIII expression precede development of diabetic complications, and thus this embodiment permits early detection of suitable patients for treatment using the methods of the invention.

As used herein, “overexpression” is any amount of apoCIII expression above control. Any suitable control can be used, including apoCIII expression levels from a subject known not to be suffering from diabetes, or previously determined standardized expression levels of apoCIII from a population of similar patient samples. Any amount of increased apoCIII expression relative to control is considered “overexpression”; in various embodiments, the overexpression comprises at least 10%, 20%, 50%, 100%, 200%, or greater increased apoCIII expression compared to control. In a preferred embodiment, apoCIII expression is detected in blood or serum samples. In one embodiment to evaluate the levels of apoCIII in sera, albumin is removed from serum samples using standard techniques, such as via use of Montage Albumin Deplete Kit (Millipore) or AlbuSorb™ (Biotech Support Group). The collected sera samples can then be freeze-dried overnight and run on sep-Pak C18. The eluted proteins can be freeze-dried and thereafter dissolved in 100 μL 0.1% TFA and run on an ACE C18 10-×0.21-cm column 20-60%, and the area under the curve, where apoCIII elutes, evaluated. ApoCIII can be identified using any suitable technique, including but not limited to MALDI mass spectrometry.

As used herein, the term “subject” or “patient” is meant any subject for which therapy is desired, including humans, cattle, dogs, cats, guinea pigs, rabbits, rats, mice, insects, horses, chickens, and so on. Most preferably, the subject is human.

The therapeutic may be administered by any suitable route, including but not limited to oral, topical, parenteral, intranasal, pulmonary, or rectal in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of the invention and a pharmaceutically acceptable carrier. The therapeutic may be present in association with one or more non-toxic pharmaceutically acceptable carriers and/or diluents and/or adjuvants, and if desired other active ingredients. The therapeutic may be in a form suitable for oral use, for example, as tablets, troches, lozenges, aqueous or oily suspensions, dispersible powders or granules, emulsion, hard or soft capsules, or syrups or elixirs.

In another embodiment, the composition can also be administered locally via implantation of a membrane, sponge, or other appropriate material onto which the composition of the invention has been absorbed or encapsulated. Where an implantation device is used, the device can be implanted into or next to the pancreas, and delivery of the desired molecule can be via diffusion, timed-release bolus, nano-containers or continuous administration.

The dosage range depends on the choice of the compound, the route of administration, the nature of the formulation, the nature of the subject's condition, and the judgment of the attending practitioner. For example, oral administration would be expected to require higher dosages than administration by intravenous injection. Variations in these dosage levels can be adjusted using standard empirical routines for optimization, as is well understood in the art

Acceptable formulation materials preferably are nontoxic to recipients at the dosages and concentrations employed. The therapeutic composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides preferably sodium or potassium chloride or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

The therapeutic compositions will be determined by a skilled artisan depending upon, for example, the intended route of administration, delivery format, and desired dosage. Such compositions can influence the physical state, stability, rate of in vivo release, and rate of in vivo clearance of the antibody-like binding protein.

### EXAMPLES

### Material And Methods

Animals. Age-matched ob/ob (Lep^{ob}/Lep^{ob}) and ob/lean (Lee^{ob}/Lep⁺) on a C57BL/6J background were obtained from a breeding colony at Karolinska Institutet, Stockholm, Sweden. Mice used in experiments were genotyped. All experiments were performed on mice, between 4 to 12 weeks of age. The animals were housed in a temperature- and humidity-controlled room with 12-hour light:dark cycles, regular chow and water ad libitum. Animal care and experimentations were carried out according to the Animal Experiment Ethics Committee at Karolinska Institutet.

Cell Culture. MIN6-m9 cells, between passage 35 and 42, were cultured in DMEM containing 11.1 mM glucose and supplemented with 100 U/ml penicillin, 100 μg/ml streptomycin, 2 mM glutamine, 10% FCS, and 75 μM β-mercaptoethanol at 5% CO₂ and 37° C. MIN6 cells were transfected using Lipofectamine (Invitrogen, USA) with the control expression construct containing tdTomato or c-Myc tagged mouse apoCIII construct for 24 hours. All experiments were performed 48 h after transfection.

Metabolic Studies. Non-fasted glucose was measured with a glucose meter (Accu-Chek™ Advantage, Roche Diagnostics, USA) and serum insulin by the ArcDia™ 2-photon fluorescence excitation microparticle fluorometry (TPX) assay for insulin (ArcDia Group, Finland).

Pancreatic islet isolation. Animals were euthanized by cervical dislocation and the pancreas perfused with 3 mL of 1 mg/mL collagenase A (Roche, USA) in Hank's balanced salt solution (HBSS) (Sigma, Sweden) buffer supplemented with 0.2% BSA and 25 mM HEPES buffer. Pancreas was thereafter extracted and digested at 37° C. for 20 min. Islets were handpicked and were either immediately used for mRNA/protein analysis or were cultured overnight in RPMI 1640 medium supplemented with 10% FCS, 2 mM glutamine as well as 100 U/ml and 100 μg/ml of penicillin and streptomycin, respectively.

Dispersion of pancreatic islets into single cells. Isolated islets were washed with HBSS without Ca²⁺ and Mg²⁺ and dispersed into single cells by incubation with Accutase in PBS containing 0.5mM EDTA (Innovative Cell Technologies, Cytotech, Denmark). Cells were kept free floating in Petri dishes in the same medium as for islets.

RNA isolation and quantitative RT-PCR analysis. Total RNA was isolated from sorted cells or isolated pancreatic islets using the RNAeasy™ Micro Kit (Qiagen, Germany). Briefly, cells were lysed by first using RLT lysis buffer followed by a spin Qiashredder™ lysis. For liver and intestine RNA isolation, tissue/cell disruption was first carried out by a hand-held rotor-stator homogenizer in RLT lysis buffer followed by 1000×g centrifugation for 5 minutes. The supernatant was subsequently transferred to a Qiashredder™ column to complete the lysis prior to the RNAeasy™ Mini Kit application (Qiagen, Germany). All lysates were applied to the RNAeasy™ spin column and the subsequent RNA isolation and on column DNAse treatment were carried out according to manufacturer recommendations. Total RNA was reverse transcribed at 37° C. with Multi scribe (Applied Biosystems, USA). The expression of all genes was measured by real-time quantitative PCR (qPCR) with Tae SYBR Supermix with ROX (Invitrogen, USA) on an ABI7900HT instrument (Applied Biosystems, USA). 13-actin was used as an endogenous control with 1 cycle at 95° C. for 10 min followed by 40 cycles of tandem 95° C. for 30 seconds and 60° C. for 1 minute. In all cases, unless otherwise stated, gene-specific intron spanning primers were used and the PCR melting curve produced one single peak corresponding to a specific single amplified product.

Western Blotting. Islets or cells were washed with PBS, lysed with RIPA buffer (50 mM Tris pH 7.5, 150 mM NaCl, 1% NP-40, 0.5% Na deoxycholate, 0.1% SDS, EDTA-free protease inhibitor cocktail, 1 μg/ml pepstatine and leupeptin). Lysates were passed 5 times through a syringe needle (0.33×13 mm/29 Gx1/2) followed by 30 minutes incubation on a rotator at 4° C. Homogenates were spun at 10,000×g for 10 minutes and the protein amount was determined in the supernatants by the BCA method (Pierce). Equal amounts of protein (25-50 μg) were separated over a 4-12% Bis-Tris gel with IVIES buffering system (Invitrogen, USA). Proteins were subsequently electrotransferred to PVDF membrane. In case of the phosphospecific antibodies, the membranes were probed with the respective antibodies and then stripped and reprobed with antibodies recognizing the respective total protein levels. Rabbit anti-FoxO1, rabbit anti-phospho-FoxO1, Akt and phospho-Akt antibody were purchased from Cell Signaling Technology (Danvers, USA). Further antibodies used were rabbit anti-apoCIII antibody (Santa-Cruz, USA), guinea-pig c-peptide (Abcam, UK), mouse anti-cMyc (Santa Cruz, USA) and mouse anti-tubulin (Abcam, UK). Immunoreactivity was detected with horseradish peroxidase-conjugated secondary antibodies using the ECL system (Amersham, USA).

Immunocytochemistry. To visualize Myc-tagged apoCIII in MIN6 cells, post-transfected cells were cultured onto glass cover slips prior to 3% PFA fixation for 30 minutes. Subsequently, cells were incubated in antibodies specific for c-Myc (mouse monoclonal, Santa-Cruz, USA) and C-peptide (rabbit polyclonal, Cell Signaling, USA) in 10% Goat serum blocking buffer, overnight at 4° C. with gentle shaking. Cells were incubated with Alexa 488 anti-mouse (Santa-Cruz, USA) and Alexa 546 anti-rabbit (Cell Signaling, USA) in blocking buffer for 1 hour followed by washing and mounting using VectaShiele™ mounting media with DAPI (Vector Labs, USA). The human islets were fixed in 4% paraformaldehyde for 15 minutes, washed in PBS and incubated in 20% sucrose/PBS overnight at 4° C. Thereafter they were embedded in Tissue-Tek® OCT™ (Sakura, The Netherlands) and cryosectioned (10 μm). The sections were permeabilized in 0.3% Triton-X/PBS for 15 minutes, treated with 3% H₂O₂ block for 10 minutes and washed in PBS and subsequently blocked in 10% goat serum. Immunohistochemical staining was performed using goat anti-human apoCIII (1:100, Academy Biomedical, USA) overnight incubation at 4° C., followed by Alexa™ Flour 488-labeled rabbit anti-goat (1:1000, Invitrogen) for 30 minutes at room temperature. Counterstaining was performed with DAPI (1:1000; Sigma-Aldrich). Following the above-described method, a blockage of goat anti-human apoCIII was also performed as a control by using the goat anti-human apoCIII pretreated over night at 4° C. with apoCIII protein (immunizing peptide).

Measurements of [Ca²⁺]ᵢ. Changes in [Ca²]ᵢ were recorded in islets after a 16 hour incubation period with 0.07 mg/ml apoCIII antisense or an inactive control (Isis Pharmaceuticals, USA) in RPMI 1640 medium. The basal medium used for islet perifusion experiments was a HEPES buffer (pH 7.4), containing: 125 mM NaCl, 5.9 mM KCl, 2.6 mM CaCl₂, 1.2 mM MgCl₂, and 25 mM HEPES, 0.1% BSA supplemented with either 3 mM glucose, 11 mM glucose or 25 mM KCl. Islets were attached to coverslips using Puramatrix™ Hydrogel (BD Biosystem, USA), loaded with 2 μM fura-2 acetoxymethyl ester (Molecular Probes, USA) and mounted on an inverted epifluorescence microscope (Zeiss, Axiovere™ 135) connected to a Spex Industries Fluorolog system for dual-wavelength excitation fluorimetry. The emissions due to the two excitation wavelengths of 340 and 380 nm were used to calculate the fluorescence ratio 340/380, reflecting changes in [Ca²⁺]ᵢ. To compensate for possible variations in output of light intensity from the two monochromators, each experiment also included measurement of a 360/360 ratio. Every experiment was normalized by dividing all fluorescence ratios by the corresponding 360/360 ratio.

Caspase assay. Activity of capase 3/7 was determined using SensoLyte™ Homogeneous Rh110 Caspase 3/7 assay kit according to manufacturer's instructions, with modifications (AnaChem, USA). Briefly, islets or cells were harvested, lysed using lysis buffer (AnaChem, USA) and the protein quantified using BCA method. 10 μg of protein was loaded in a black 384-well plate and topped up with the appropriate amount of assay buffer (AnaChem, USA) containing Rh110 Caspase 3/7 Substrate. Plate was incubated in the dark for 1 hour at 24° C. Fluorescence intensity at Ex/Em=490/520nm was measured to determine the relative caspase activity.

RNA analysis. 5′-RACE was performed by using ob/ob islets (1.5 μg RNA) Ambion FirstChoice™ RACE-ready cDNA kit (Invitrogen, USA). PCR was carried-out following manufacturer's instructions. Platinum Taq polymerase (Invitrogen, USA) was used in RACE-PCR. Primer sequences for apoCIII-outer (primary amplification): 5′ GGAGGGGTGAAGACATGAGA-3′ (SEQ ID NO: 70); apoCIII-inner (nested amplification): 5′ TCTGAAGTGATTGTCCATCCAG-3′ (SEQ ID NO: 71). An aliquot of the first PCR (outer PCR) was used for subsequent nested PCR (inner PCR). Amplified cDNA were gel purified and subcloned into the pCRII-TOPO vector (Invitrogen, USA) and sequenced with M13 reverse and M13 forward primers.

Statistical analysis. Studies were repeated at least three times. For individual experiment, the number of animals used (n) is included in each figure legend in parenthesis. All results are expressed as mean±SEM (indicated by error bars). Statistical analyzes were performed with either GraphPad™ Prism 5 or Microsoft Excel 2007. A student's t-test or one-way ANOVA (Tukey's post-hoc) were used as appropriate. P values <0.05 were considered statistically significant.

### Example 1

### Insulin Signaling in ob/ob Islets

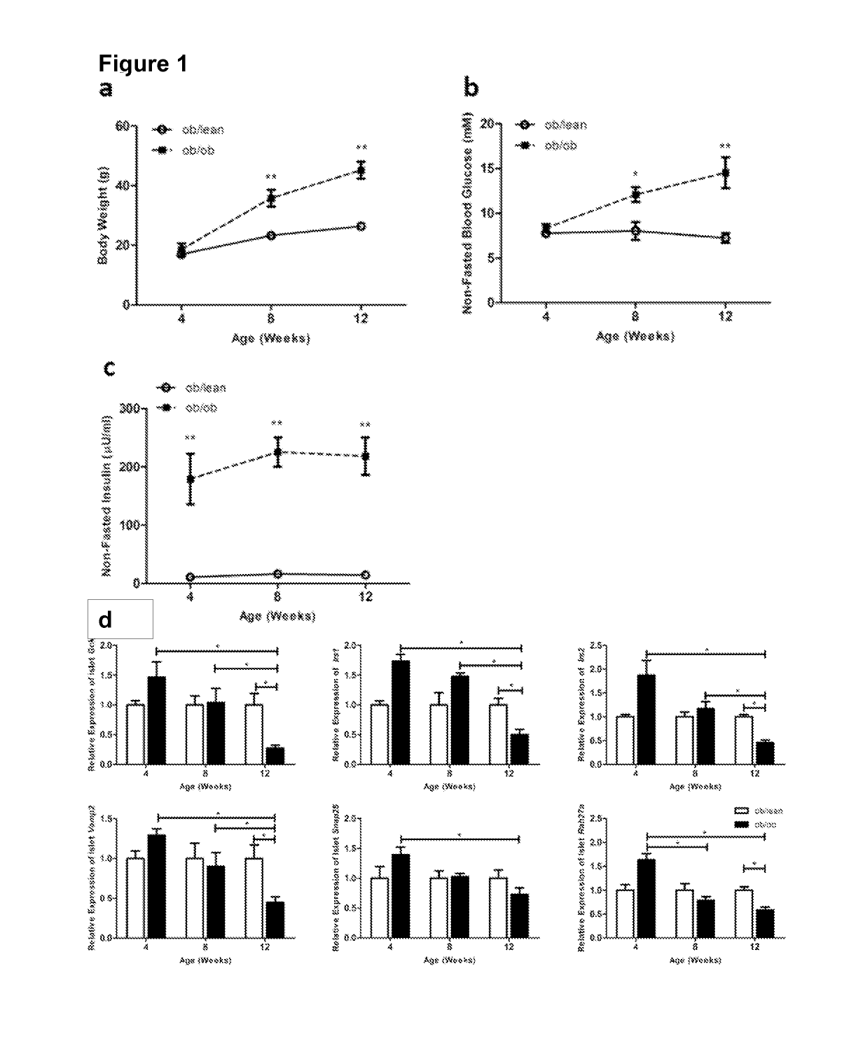

To determine the changes in insulin signaling in ob/ob mouse islets, which consist of more than 90% β-cells, the phenotypic changes were tracked in these mice at 4, 8 and 12 weeks of age, measuring parameters such as body weight, non-fasting blood glucose and insulin concentrations. Age-matched ob/lean mice that were routinely genotyped from 4 weeks onwards were used as controls. Since the ob/ob mouse is a known transient hyperglycemia model with increasing hyperglycemia up to 14 weeks of age, the study was limited to the first 12 weeks of age and found that both weight and non-fasting blood glucose levels progressively increased from 4- to 12-weeks of age (FIG. 1a and FIG. 1b). However, the non-fasting insulin levels in the ob/ob mice remained higher than in control animals (FIG. 1c).

With prolonged hyperinsulinemia, insulin signaling at the islet level may be compromised, similar to what is seen in the β-cell-specific insulin receptor knockout mouse (βIRKO) and the β-cell-specific PI3K subunit p85 β knockout mouse (βPik3r1). The mRNA expression levels of several genes known to be involved downstream of islet insulin signaling was determined. The expression of gck, irs1, irs2, vamp2, snap25 and rab27a were shown to be controlled by insulin receptor (IR) activated phosphatidylinositol 3-kinase (PI3K) activity. All genes were down-regulated in 12 weeks old ob/ob islets (FIG. 1d). This is consistent with previous findings where a significantly reduced PI3K activity was found in 12-weeks old ob/ob islets as compared with their age-matched ob/lean littermates.

### Example 2

### Progressive Change of FoxO1-Regulated Gene Expression in ob/ob Islets Over Time

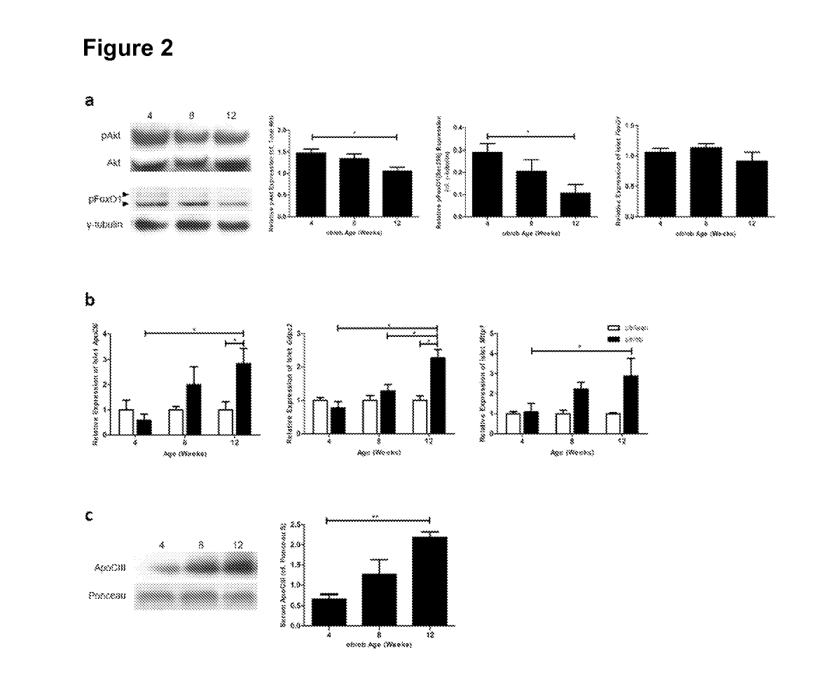

One of the ways in which insulin induces changes in gene expression is through the phosphorylation cascade of Akt and its downstream target forkhead transcription factor FoxO1. Insulin-mediated activation of Akt leads to the phosphorylation of FoxO1 with its nuclear exclusion and loss of transcriptional activity. This signaling cascade has been shown to be crucial in maintaining β-cell function and proliferation during either insulin resistance or insult/injury. Levels of phospho-Akt and phospho-FoxO1 in ob/ob islets were determined at 4-, 8- and 12-weeks of age. There was a significant reduction in phospho-Akt (Ser473) and phospho-FoxO1 (Ser256) in the 12-weeks old ob/ob islet as compared to the islets at 4-weeks of age (FIG. 2a). Total Akt protein levels within these islets were not significantly different across time. Messenger RNA levels of FoxO1 were determined, and found no significant difference in all the age groups studied. The reduction in phospho-FoxO1 further corroborates the changes in ob/ob islet gene expression since irs1, irs2, vamp2 and rab27a have been suggested to be repressed by FoxO1.

Insulin receptor-mediated repression of FoxO1 is pivotal in maintaining the balance between carbohydrate and fat metabolism in hepatocytes. Reduced FoxO1 phosphorylation, and subsequent activation, in insulin-resistant hepatocytes promote the expression of apoCIII, a modulator of circulating triglycerides, glucose 6 phosphatase a key gluconeogenic enzyme, and mttp1, a microsomal triglyceride transfer protein that is involved in lipoprotein assembly. To explore if a similar mechanism occurs in ob/ob islets with declining levels of phospho-FoxO1, the mRNA expression levels of Foxo1 target genes apoCIII, g6pc2 (islet specific glucose-6-phosphatase) and mttp1 were determined. There was a significant increase in ob/ob islet mRNA expression of all three genes across time (FIG. 2b). Together, these data suggest that insulin resistance at the islet level leads to a change in the expression of genes that are positively or negatively regulated by FoxO1. Of these, apoCIII expression stands out as a novel islet factor that has been shown to disrupt β-cell function. G6pc2 was originally identified as a variant in

T2DM genome wide association (GWA) studies but its function did not appear to translate into an increased risk of T2DM.

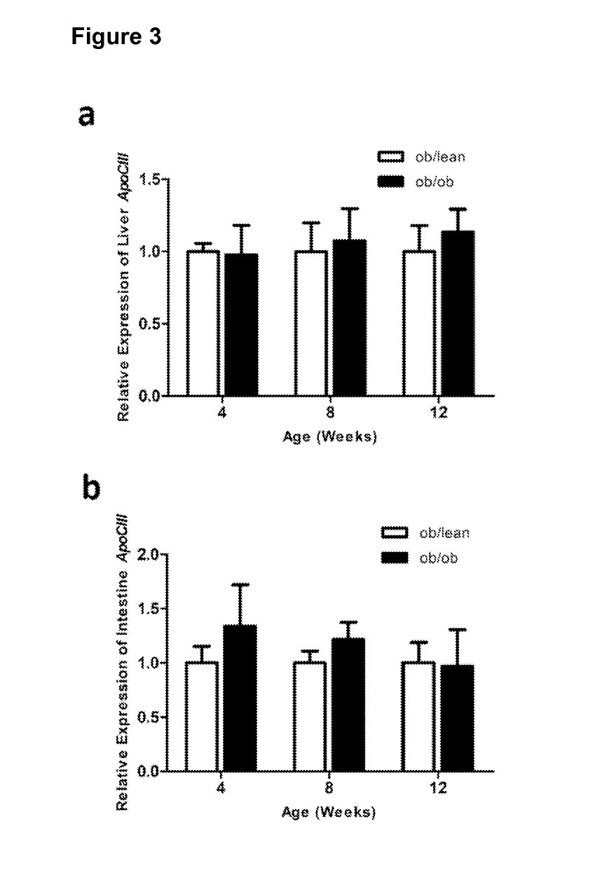

There was no significant difference in liver and intestine apoCIII expression in ob/ob mice at the age-groups studied (FIG. 3). The systemic levels of apoCIII in ob/ob mice were also examined, and it was surprisingly discovered that the apoCIII immunoblots showed increasing apoCIII levels in the serum of ob/ob mice from 4 to 12 weeks of age (FIG. 1c). As liver apoCIII accounts for the bulk of circulating apoCIII, the higher levels of circulating apoCIII could be the result of reduced cellular uptake of triglyceride-rich (VLDL) remnants rather than increased production of VLDL particles.

### Example 3

### ApoCIII is Detected in Both ob/ob Mice and Human Pancreatic Islets

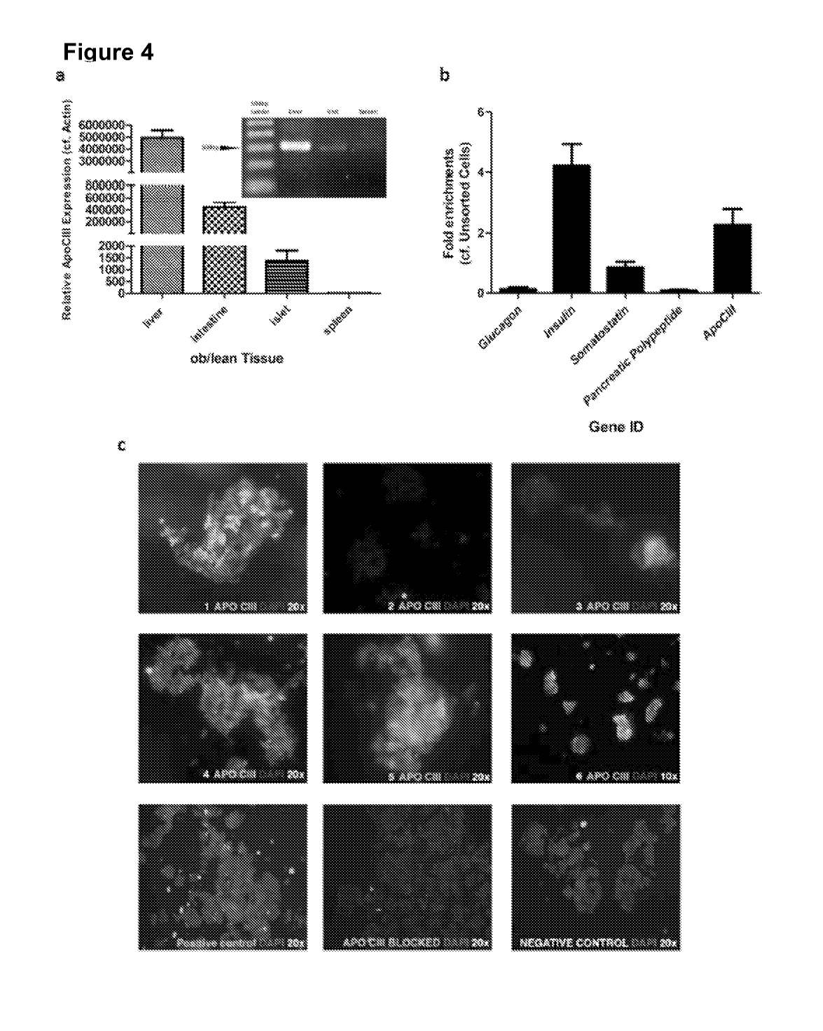

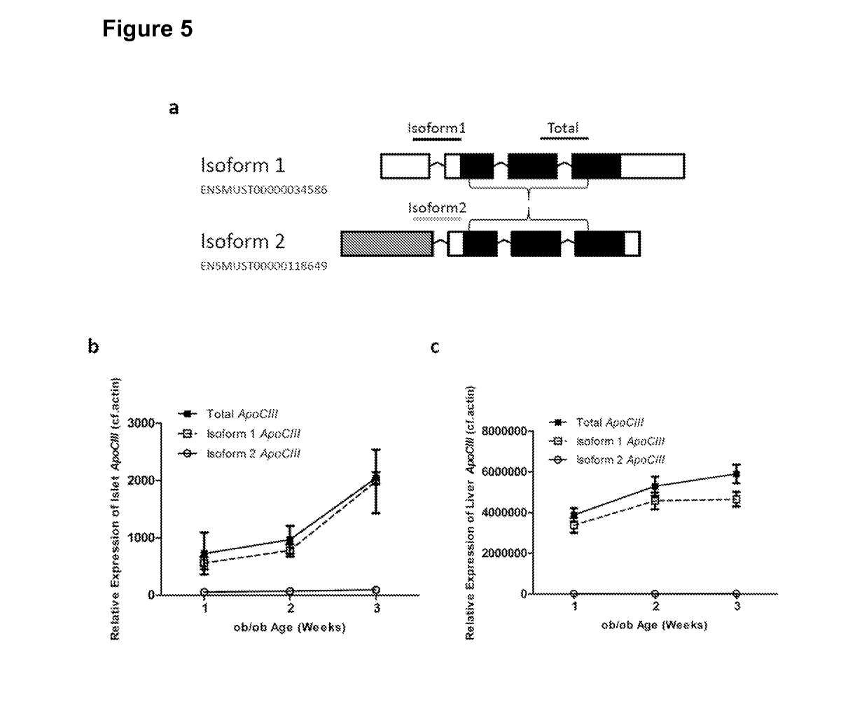

The presence of apoCIII in pancreatic islet has not been observed before and is interesting since this apolipoprotein is known to be involved in both β-cell dysfunction and T1DM. A proteomic characterization of single pancreatic islets showed presence of apoCIII. However, this can be the result of either systemic circulating apoCIII residing within the microvasculature of pancreatic islets or, as shown here, a local production of apoCIII within the islet itself Comparatively, the expression of apoCIII in ob/lean pancreatic islets is about 3000-fold lower compared to the liver (FIG. 4a). The small intestine, the other known organ expressing apoCIII, has an expression that is 10-fold lower compared to the liver, but 300-fold higher compared to islet apoCIII expression. Nevertheless, a three-fold increase in apoCIII mRNA seen in the 12-weeks old ob/ob islet may exert profound autocrine/paracrine effects within the islet (FIG. 2b). A 5′RACE of apoCIII transcripts in islets of 12-weeks old ob/ob mice confirmed the presence of two transcripts with a differing 5′-untranslated region and a 100% identical coding region (FIG. 5). The predominant islet apoCIII transcript is the same transcript that is expressed in the liver, suggesting that the same promoter is governing apoCIII transcription in the liver and in islet cells.

The source of apoCIII within the islets was determined. The endocrine portion of the ob/ob islet is made up of approximately 90% β-cells, making this cell type a prime candidate for apoCIII expression. To examine this, a flow cytometry protocol originally optimized to sort out rat β-cells from the total pancreatic islet cell milieu was used. Subsequent characterization of sorted β-cells revealed enriched expression of insulin, low expression of somatostatin and undetectable expression of glucagon and pancreatic polypeptide (FIG. 4b). The expression of apoCIII was similarly higher, albeit lower compared to insulin, in the β-cell enriched population strongly suggesting that apoCIII is expressed in the pancreatic β-cell (FIG. 4b). Immunofluorescence staining using human apoCIII antibodies revealed the presence of apoCIII in human islet preparations from five donors, three males and two females, with no history of diabetes (FIG. 4c). Hence, these data demonstrate that the local production of apoCIII in pancreatic islets is conserved from rodents to humans.

### Example 4

### ApoCIII Overexpression Increases [Ca²⁺]ᵢ in MIN6 Cells

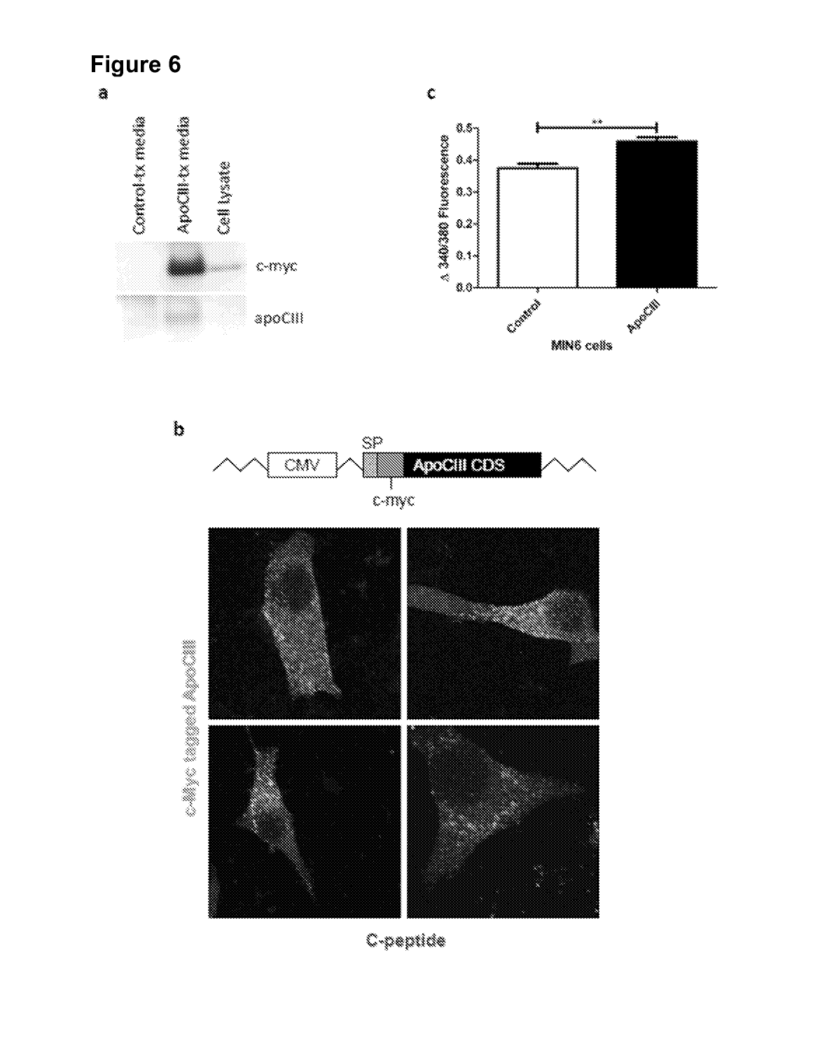

Exogenously added human apoCIII induces apoptosis in the rat insulin secreting cells lines RINm5F and INS-1E and in primary mouse β-cells. It was therefore important to determine if a local production of apoCIII likewise affected mouse β-cell function and apoptosis. Hampered by the ability of the antibody to recognize apoCIII in mouse liver sections and lysates, a c-Myc tagged mouse apoCIII construct was used that allows determining the localization of Myc-apoCIII within β-cells. Myc-apoCIII was detectable in media of the Myc-apoCIII-transfected cells after a 24-hour culture, indicating that the synthesized apoCIII is being released into the extracellular medium (FIG. 6a). No co-localization between Myc-apoCIII and insulin C-peptide could be detected in the transfected cells (FIG. 6b), suggesting that apoCIII is consecutively released from the β-cell and not co-released with insulin. MIN6 cells transfected with either Myc-apoCIII or tdTomato (control vector) were loaded with Fura-2AM after 48 hours to determine the effect of mouse apoCIII on [Ca²⁺]ᵢ upon KCl stimulation. An increase in [Ca²⁺]ᵢ after KCl exposure was observed in MIN6 cells expressing Myc-apoCIII as compared to the control transfected cells (FIG. 6c). This was consistent with previous effects of exogenously added human apoCIII on both rat insulinoma cells and primary β-cells, indicating a conservation of function between human apoCIII and mouse apoCIII despite only having a 60% amino acid identity. The transfection efficiency was about 50%, but a higher increase in [Ca²⁺]ᵢ, upon depolarization was seen in non-transfected cells in the same culture dish, and the levels were above those from control transfected cells. This suggests that the non-transfected cells were affected by the apoCIII released into the media from neighboring Myc-apoCIII expressing cells.

### Example 5

### Reduced Endogenous Islet apoCIII Results in Improved [Ca²⁺]ᵢ Handling and Less Apoptosis

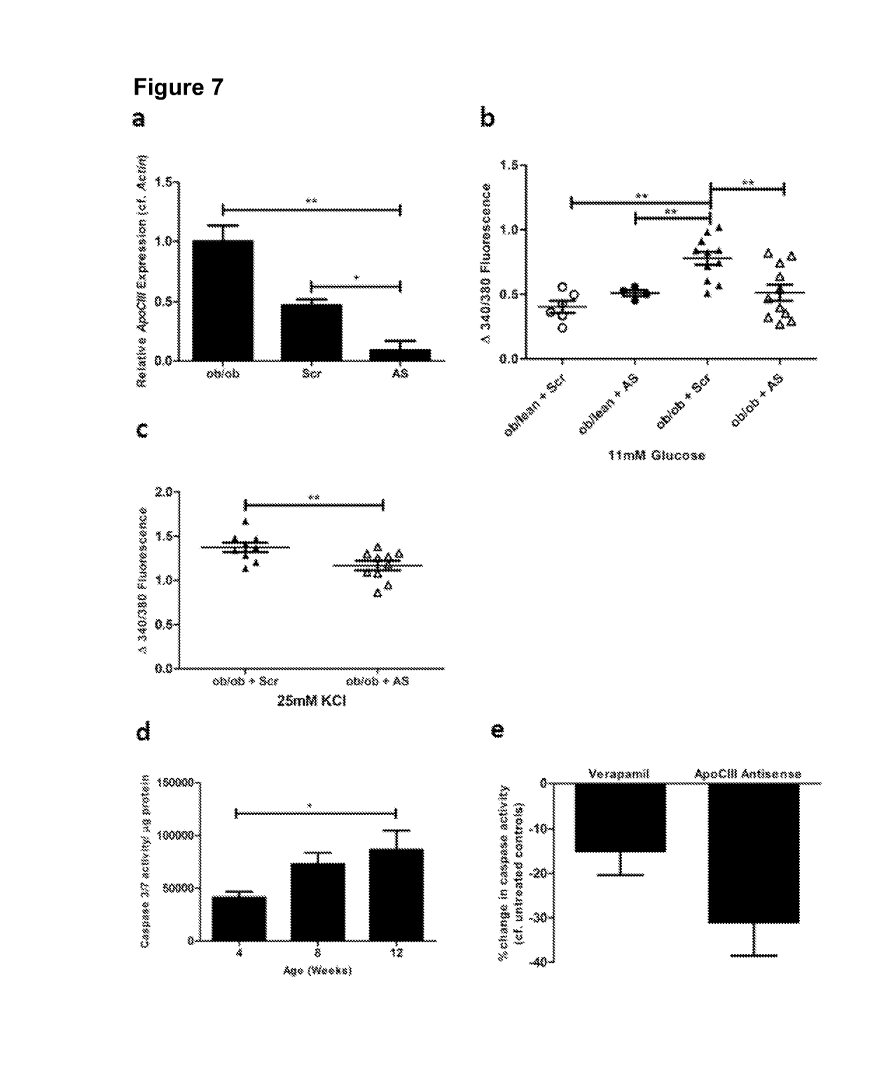

To explore the effect of increased endogenous apoCIII expression within islets from 12-weeks old ob/ob mice, knockdown experiments using antisense oligonucleotides specific for apoCIII and an inactive control were performed. The expression of apoCIII in the islets exposed to antisense nucleotides for 18-hours was 20% compared to control islets (FIG. 7a). The overall expression of apoCIII in pancreatic islets fell to about 50% just after 18 hours in culture compared to mRNA levels of apoCIII in freshly isolated islets, suggesting that an artificial milieu associated with islet culture. Subsequently pancreatic islets were loaded with Fura-2AM to determine the effect of apoCIII on [Ca²⁺]ᵢ after both high glucose and KCl exposure. Ob/ob islets were more responsive to high glucose concentrations, as compared to ob/lean islets, with a significantly higher peak amplitude of [Ca²⁺]ᵢ (FIG. 7b). The knockdown of apoCIII in 12-weeks old ob/ob islets reduced the peak amplitude of [Ca²⁺]ᵢ upon both high glucose and KCl stimulation (FIG. 7b, 7c).

It was determined if reducing endogenous apoCIII affects apoptosis in ob/ob islets. Although ob/ob islets are hyperplastic with massive β-cell proliferation, it does not rule out the presence of apoptosis in these islets; therefore the level of apoptosis was determined in freshly isolated islets of ob/ob mice at weeks 4-, 8- and 12 (when phospho-FoxO1 declines) by measuring the activity of caspase 3/7. Apoptosis in ob/ob islets was significantly higher in 12-weeks old ob/ob islets as compared to islets at 4 weeks of age (FIG. 7d). Lowering endogenous apoCIII in 12-weeks-old ob/ob islets, when apoCIII levels are the highest, decreased caspase 3/7 activity, as a measure of apoptosis, by 31% compared to islets treated with scrambled antisense (FIG. 7e). Previous results have shown that apoCIII increases the activity of the voltage-gated Ca²⁺-channel. Exposing β-cells to the Ca²⁺-channel blocker verapamil reduced caspase activity by 15% (FIG. 7e), suggesting that increased FoxO1-induced expression of apoCIII within the ob/ob pancreatic islet leads to unbalanced [Ca²⁺]ᵢ and an increase in β-cell apoptosis.

These results show that obesity is associated with a local production of apoCIII in pancreatic β-cells and that increased levels of apoCIII leads to insulin resistance, increased [Ca2+]ᵢ and β-cell apoptosis; therefore identifying local islet production of apoCIII as a novel factor for mediating β-cell apoptosis in insulin resistant states of T2DM. The effects are reversible as reducing endogenouse β-cell apoCIII normalizes [Ca2+]ᵢ and improves β-cell survival. Hence, locally enhanced apoCIII can interfere with β-cell function and survival and have an impact on development of the T2DM phenotype. The results demonstrate that apoCIII is locally expressed and increased in islets from ob/ob mice that have reduced insulin signaling. As these islets contain >90% β-cells, most of the apoCIII expression is derived from the insulin-secreting β-cells. ApoCIII induced an increase in cytoplasmic free Ca2+ concentration and β-cell apoptosis that was abrogated by lowering apoCIII levels or blocking the voltage-gated Ca2+-channels. This makes apoCIII a prime target that links β-cell insulin resistance to reduction in β-cell mass in T2DM. Consequently, inhibition of β-cell apoCIII expression constitutes a novel mechanism to maintain β-cell function and survival.

In summary, insulin signaling in β-cells is essential to keep apoCIII expression low. When insulin signaling is impaired at the islet level, FoxO1 translocation into the nucleus induces changes in gene expression which include, among other alterations, a promotion of apoCIII transcription. The increase in local production of apoCIII subsequently leads to an increase in [Ca²⁺]ᵢ which in turn results in an increase in β-cell apoptosis. Hence, preventing insulin resistance at the islet level is crucial to preserve β-cell mass, especially during the late phase of T2DM where β-cells are progressively lost. Islet apoCIII is a key player in the promotion of β-cell apoptosis and lowering local β-cell production of apoCIII may provide a new therapeutic for T2DM.

### REFERENCES

Martin, B. C., et al. Role of glucose and insulin resistance in development of type 2 diabetes mellitus: results of a 25-year follow-up study. Lancet 340, 925-929 (1992).
Lillioja, S., et al. Insulin resistance and insulin secretory dysfunction as precursors of non-insulin-dependent diabetes mellitus. Prospective studies of Pima Indians. The New England journal of medicine 329, 1988-1992 (1993).
Kahn, C. R. Banting Lecture. Insulin action, diabetogenes, and the cause of type II diabetes. Diabetes 43, 1066-1084 (1994).
Leibiger, I. B., Leibiger, B., Moede, T. & Berggren, P. O. Exocytosis of insulin promotes insulin gene transcription via the insulin receptor/PI-3 kinase/p70 s6 kinase and CaM kinase pathways. Molecular cell 1, 933-938 (1998).
Kulkarni, R. N., et al. Tissue-specific knockout of the insulin receptor in pancreatic beta cells creates an insulin secretory defect similar to that in type 2 diabetes. Cell 96, 329-339 (1999).
Kulkarni, R. N., et al. Altered function of insulin receptor substrate-1-deficient mouse islets and cultured beta-cell lines. J Clin Invest 104, R69-75 (1999).
Okada, T., et al. Insulin receptors in beta-cells are critical for islet compensatory growth response to insulin resistance. Proceedings of the National Academy of Sciences of the United States of America 104, 8977-8982 (2007).
Ueki, K., et al. Total insulin and IGF-I resistance in pancreatic beta cells causes overt diabetes. Nature genetics 38, 583-588 (2006).
Hashimoto, N., et al. Ablation of PDK1 in pancreatic beta cells induces diabetes as a result of loss of beta cell mass. Nature genetics 38, 589-593 (2006).
Kaneko, K., et al. Class IA phosphatidylinositol 3-kinase in pancreatic beta cells controls insulin secretion by multiple mechanisms. Cell metabolism 12, 619-632 (2010).
Krauss, R. M., Herbert, P. N., Levy, R. I. & Fredrickson, D. S. Further observations on the activation and inhibition of lipoprotein lipase by apolipoproteins. Circ Res 33, 403-411(1973).
Ginsberg, H. N., et al. Apolipoprotein B metabolism in subjects with deficiency of apolipoproteins CIII and Al. Evidence that apolipoprotein CIII inhibits catabolism of triglyceride-rich lipoproteins by lipoprotein lipase in vivo. J Clin Invest 78, 1287-1295 (1986).
Chen, M., Breslow, J. L., Li, W. & Leff, T. Transcriptional regulation of the apoC-III gene by insulin in diabetic mice: correlation with changes in plasma triglyceride levels. J Lipid Res 35, 1918-1924 (1994).
Altomonte, J., et al. Foxol mediates insulin action on apoC-III and triglyceride metabolism. J Clin Invest 114, 1493-1503 (2004).
Caron, S., et al. Transcriptional activation of apolipoprotein CIII expression by glucose may contribute to diabetic dyslipidemia. Arteriosclerosis, thrombosis, and vascular biology 31, 513-519 (2011).
Salerno, A. G., et al. Overexpression of apolipoprotein CIII increases and CETP reverses diet-induced obesity in transgenic mice. Int J Obes (Lond) 31, 1586-1595 (2007).
Waterworth, D. M., et al. ApoCIII gene variants modulate postprandial response to both glucose and fat tolerance tests. Circulation 99, 1872-1877 (1999).
Petersen, K. F., et al. Apolipoprotein C3 gene variants in nonalcoholic fatty liver disease. The New England Journal of Medicine 362, 1082-1089 (2010).
Juntti-Berggren, L., et al. Apolipoprotein CIII promotes Ca2+-dependent beta cell death in type 1 diabetes. Proceedings of the National Academy of Sciences of the United States of America 101, 10090-10094 (2004).
Refai, E., et al. Transthyretin constitutes a functional component in pancreatic beta-cell stimulus-secretion coupling. Proceedings of the National Academy of Sciences of the United States of America 102, 17020-17025 (2005).
Holmberg, R., et al. Lowering apolipoprotein CIII delays onset of type 1 diabetes. Proceedings of the National Academy of Sciences of the United States of America 108, 10685-10689 (2011).
Hellman, B. Studies in obese-hyperglycemic mice. Annals of the New York Academy of Sciences 131, 541-558. (1965).
Coleman, D. L. & Hummel, K. P. The influence of genetic background on the expression of the obese (Ob) gene in the mouse. Diabetologia 9, 287-293 (1973).
Huo, L., et al. Leptin-dependent control of glucose balance and locomotor activity by POMC neurons. Cell metabolism 9, 537-547 (2009).
Kieffer, T. J., Heller, R. S., Leech, C. A., Holz, G. G. & Habener, J. F. Leptin suppression of insulin secretion by the activation of ATP-sensitive K+ channels in pancreatic beta-cells. Diabetes 46, 1087-1093 (1997).
Laubner, K., et al. Inhibition of preproinsulin gene expression by leptin induction of suppressor of cytokine signaling 3 in pancreatic beta-cells. Diabetes 54, 3410-3417 (2005).
Leibiger, B., et al. Insulin-feedback via PI3K-C2alpha activated PKBalpha/Akt1 is required for glucose-stimulated insulin secretion. FASEB journal: official publication of the Federation of American Societies for Experimental Biology 24, 1824-1837 (2010).
Kerouz, N. J., Horsch, D., Pons, S. & Kahn, C. R. Differential regulation of insulin receptor substrates-1 and -2 (IRS-1 and IRS-2) and phosphatidylinositol 3-kinase isoforms in liver and muscle of the obese diabetic (ob/ob) mouse. J Clin Invest 100, 3164-3172 (1997).
Zawalich, W. S., Tesz, G. J. & Zawalich, K. C. Inhibitors of phosphatidylinositol 3-kinase amplify insulin release from islets of lean but not obese mice. The Journal of endocrinology 174, 247-258 (2002).
Kitamura, T., et al. The forkhead transcription factor Foxol links insulin signaling to Pdxl regulation of pancreatic beta cell growth. J Clin Invest 110, 1839-1847 (2002).
Kitamura, Y. I., et al. FoxO1 protects against pancreatic beta cell failure through NeuroD and MafA induction. Cell metabolism 2, 153-163 (2005).
Okamoto, H., et al. Role of the forkhead protein FoxO1 in beta cell compensation to insulin resistance. J Clin Invest 116, 775-782 (2006).
Kamagate, A., et al. FoxO1 mediates insulin-dependent regulation of hepatic VLDL production in mice. J Clin Invest 118, 2347-2364 (2008).
Chen, W. M., et al. Variations in the G6PC2/ABCB11 genomic region are associated with fasting glucose levels. J Clin Invest 118, 2620-2628 (2008).
Wiegman, C. H., et al. Hepatic VLDL production in ob/ob mice is not stimulated by massive de novo lipogenesis but is less sensitive to the suppressive effects of insulin. Diabetes 52, 1081-1089 (2003).
Sol, E. M., Sundsten, T. & Bergsten, P. Role of MAPK in apolipoprotein CIT-induced apoptosis in INS-1E cells. Lipids in health and disease 8, 3 (2009).
Waanders, L. F., et al. Quantitative proteomic analysis of single pancreatic islets. Proceedings of the National Academy of Sciences of the United States of America 106, 18902-18907 (2009).
Kohler, M., et al. One-step purification of functional human and rat pancreatic alpha cells. Integrative biology: quantitative biosciences from nano to macro 4, 209-219 (2012).
Kluth, O., et al. Dissociation of lipotoxicity and glucotoxicity in a mouse model of obesity associated diabetes: role of forkhead box O1 (FOXO1) in glucose-induced beta cell failure. Diabetologia 54, 605-616 (2011).
Kulkarni, R. N., et al. beta-cell-specific deletion of the Igfl receptor leads to hyperinsulinemia and glucose intolerance but does not alter beta-cell mass. Nature genetics 31, 111-115 (2002).
Xuan, S., et al. Defective insulin secretion in pancreatic beta cells lacking type 1 IGF receptor. J Clin Invest 110, 1011-1019 (2002).
Hribal, M. L., Nakae, J., Kitamura, T., Shutter, J. R. & Accili, D. Regulation of insulin-like growth factor-dependent myoblast differentiation by Foxo forkhead transcription factors. The Journal of cell biology 162, 535-541 (2003).
Kubota, N., et al. Disruption of insulin receptor substrate 2 causes type 2 diabetes because of liver insulin resistance and lack of compensatory beta-cell hyperplasia. Diabetes 49, 1880-1889 (2000).
Leibiger, I. B., Leibiger, B. & Berggren, P. O. Insulin signaling in the pancreatic beta-cell. Annual review of nutrition 28, 233-251 (2008).
Sakuraba, H., et al. Reduced beta-cell mass and expression of oxidative stress-related DNA damage in the islet of Japanese Type II diabetic patients. Diabetologia 45, 85-96 (2002).
Butler, A. E., et al. Beta-cell deficit and increased beta-cell apoptosis in humans with type 2 diabetes. Diabetes 52, 102-110 (2003).
Donath, M. Y., et al. Mechanisms of beta-cell death in type 2 diabetes. Diabetes 54 Suppl 2, S108-113 (2005).
Martinez, S. C., Cras-Meneur, C., Bernal-Mizrachi, E. & Permutt, M. A. Glucose regulates Foxol through insulin receptor signaling in the pancreatic islet beta-cell. Diabetes 55, 1581-1591 (2006).
Chan, D. C., Nguyen, M. N., Watts, G. F. & Barrett, P. H. Plasma apolipoprotein C-III transport in centrally obese men: associations with very low-density lipoprotein apolipoprotein B and high-density lipoprotein apolipoprotein A-I metabolism. The Journal of clinical endocrinology and metabolism 93, 557-564 (2008).
Joven, J., et al. Concentrations of lipids and apolipoproteins in patients with clinically well-controlled insulin-dependent and non-insulin-dependent diabetes. Clin Chem 35, 813-816 (1989).
al Muhtaseb, N., al Yousuf, A. & Bajaj, J. S. Apolipoprotein A-I, A-II, B, C-II, and C-III in children with insulin-dependent diabetes mellitus. Pediatrics 89, 936-941 (1992).
Stewart, M. W., Laker, M. F. & Alberti, K. G. The contribution of lipids to coronary heart disease in diabetes mellitus. J Intern Med Suppl 736, 41-46 (1994).
Hiukka, A., et al. Alterations of lipids and apolipoprotein CIII in very low density lipoprotein subspecies in type 2 diabetes. Diabetologia 48, 1207-1215 (2005).
Sundsten, T., Zethelius, B., Berne, C. & Bergsten, P. Plasma proteome changes in subjects with Type 2 diabetes mellitus with a low or high early insulin response. Clinical science 114, 499-507 (2008).
Hokanson, J. E., et al. Susceptibility to type 1 diabetes is associated with ApoCIII gene haplotypes. Diabetes 55, 834-838 (2006).
Pollin, T. I., et al. A null mutation in human APOC3 confers a favorable plasma lipid profile and apparent cardioprotection. Science 322, 1702-1705 (2008).
Atzmon, G., et al. Lipoprotein genotype and conserved pathway for exceptional longevity in humans. PLoS biology 4, e113 (2006).
Duivenvoorden, I., et al. Apolipoprotein C3 deficiency results in diet-induced obesity and aggravated insulin resistance in mice. Diabetes 54, 664-671 (2005).
Lee, H. Y., et al. Apolipoprotein CIII overexpressing mice are predisposed to diet-induced hepatic steatosis and hepatic insulin resistance. Hepatology 54, 1650-1660 (2011).
Kawakami, A., et al. Apolipoprotein CIII induces expression of vascular cell adhesion molecule-1 in vascular endothelial cells and increases adhesion of monocytic cells. Circulation 114, 681-687 (2006).
Hiukka, A., et al. ApoCIII-enriched LDL in type 2 diabetes displays altered lipid composition, increased susceptibility for sphingomyelinase, and increased binding to biglycan. Diabetes 58, 2018-2026 (2009).

We claim:

1. A method for treating or limiting development of diabetes, comprising administering to a subject in need thereof an amount effective of a composition comprising a compound of formula A-B, wherein A is a pancreatic β cell targeting moiety, and B is an inhibitor of expression and/or activity of Apolipoprotein CIII (apoCIII), protein kinase A (PKA), Src kinase, and/or β1 integrin.

2. The method of claim 1, wherein the pancreatic β cell specific targeting moiety comprises a moiety that that selectively binds a protein selected from the group consisting of DiGeorge syndrome critical region gene 2 (DGCR2), golgi brefeldin A resistant guanine nucleotide exchange factor 1 (GBF1), orphan G protein-coupled receptor GPR44(GPR44), SerpinB10 (PI-10), FXYD domain containing ion transport regulator 2 (FXYD2), Tetraspanin-7 (TSPAN7), gap junction protein, delta 2, 36 kDa (GJD2), solute carrier family 18 (vesicular monoamine), member 2 (SLC18A2), prokineticin receptor 1 (PROKR1), glutamate receptor, metabotropic 5 (GRM5), neuropeptide Y receptor Y2 (NPY2R), glucagon-like peptide 1 receptor (GLP1R), and transmembrane protein 27 (TMEM27).

3. The method of claim 1, wherein the pancreatic β cell specific targeting moiety comprises an antibody or an aptamer.

4. The method of claim 1, wherein the pancreatic β cell specific targeting moiety comprises a moiety selected from the group consisting of glucagon-like peptide-1 (GLP-1), glucagon-like peptide-2 (GLP-2), peptide YY (PYY), neuropeptide Y (NPY), pancreatic peptide (PP), exendin-4, naphthylalanine and naphthylalanine derivatives.

5. The method of claim 1, wherein the inhibitor comprises an apoCIII inhibitor.

6. The method of claim 5, wherein the apoCIII inhibitor is selected from the group consisting of anti-apoCIII antibody, anti-apoCIII aptamer, apoCIII small interfering RNA, apoCIII small internally segmented interfering RNA, apoCIII short hairpin RNA, apoCIII microRNA, and apoCIII antisense oligonucleotides.

7. The method of claim 1, wherein the method comprises administering the composition to a subject that overexpresses apoCIII relative to control.

8. The method of claim 1, wherein the subject has or is at risk of developing type 1 diabetes.

9. The method of claim 1, wherein the subject has or is at risk of developing type 2 diabetes.

10. The method of claim 1, wherein the subject has type 2 diabetes.

11. A composition of formula A-B, wherein A is a pancreatic β cell targeting moiety, and B is an inhibitor of Apolipoprotein CIII (apoCIII), protein kinase A (PKA), Src kinase, or β1 integrin.

12. The composition of claim 11, wherein the pancreatic β cell specific targeting moiety comprises a moiety that that selectively binds a protein selected from the group consisting of DiGeorge syndrome critical region gene 2 (DGCR2), golgi brefeldin A resistant guanine nucleotide exchange factor 1 (GBF1), orphan G protein-coupled receptor GPR44(GPR44), SerpinB10 (PI-10), FXYD domain containing ion transport regulator 2 (FXYD2), Tetraspanin-7 (TSPAN7), gap junction protein, delta 2, 36 kDa (GJD2), solute carrier family 18 (vesicular monoamine), member 2 (SLC18A2), prokineticin receptor 1 (PROKR1), glutamate receptor, metabotropic 5 (GRM5), neuropeptide Y receptor Y2 (NPY2R), glucagon-like peptide 1 receptor (GLP1R), and transmembrane protein 27 (TMEM27).

13. The composition of claim 11, wherein the pancreatic β cell specific targeting moiety comprises an antibody or an aptamer

14. The composition of claim 11, wherein the pancreatic β cell specific targeting moiety comprises a moiety selected from the group consisting of glucagon-like peptide-1 (GLP-1), glucagon-like peptide-2 (GLP-2), peptide YY (PYY), neuropeptide Y (NPY), pancreatic peptide (PP), exendin-4, naphthylalanine and naphthylalanine derivatives

15. The composition of claim 11, wherein the inhibitor comprises an ApoCIII inhibitor.

16. The composition of claim 15, wherein the inhibitor is selected from the group consisting of anti-apoCIII antibody, anti-apoCIII aptamer, apoCIII small interfering RNA, apoCIII small internally segmented interfering RNA, apoCIII short hairpin RNA, apoCIII microRNA, and apoCIII antisense oligonucleotides.

17. The composition of claim 11, wherein the composition is of formula A-B-C, wherein C is a compound that permits cell entry of the composition.

18. The composition of claim 17, wherein C is a cell penetrating peptide.

19. The composition of claim 11, wherein the composition comprises a fusion protein.

20. An isolated nucleic acid encoding the fusion protein of claim 19 operatively linked to a promoter.

21. A recombinant expression vector comprising the isolated nucleic acid of claim 20.

22. An isolated recombinant host cell comprising the expression vector of claim 21.


SEQUENCE LISTING (76 sequences)

SEQ ID NO: 1 - protein, 180 aa, Artificial Sequence
  Synthetic
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 2 - skipped

SEQ ID NO: 3 - protein, 97 aa, Artificial Sequence
  Synthetic
MVFVRRPWPALTTVLLALLVCLGALVDAYPIKPEAPGEDASPEELNRYYASLRHYLNLVTRQRYGKRDGPDTLLSKTFFPDGEDRPVRSRSEGPDLW

SEQ ID NO: 4 - protein, 97 aa, Artificial Sequence
  Synthetic
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 5 - protein, 95 aa, Artificial Sequence
  Synthetic
MAAARLCLSLLLLSTCVALLLQPLLGAQGAPLEPVYPGDNATPEQMAQYAADLRRYINMLTRPRYGKRHKEDTLAFSEWGSPHAAVPRELSPLDL

SEQ ID NO: 6 - protein, 87 aa, Artificial Sequence
  Synthetic
MKIILWLCVFGLFLATLFPISWQMPVESGLSSEDSASSESFASKIKRHGEGTFTSDLSKQMEEEAVRLFIEWLKNGGPSSGAPPPSG

SEQ ID NO: 7 - protein, 550 aa, Artificial Sequence
  Synthetic
MVPKADSGAFLLLFLLVLTVTEPLRPELRCNPGQFACRSGTIQCIPLPWQCDGWATCEDESDEANCPEVTGEVRPHHGKEAVDPRQGRARGGDPSHFHAVNVAQPVRFSSFLGKCPTGWHHYEGTASCYRVYLSGENYWDAAQTCQRLNGSLATFSTDQELRFVLAQEWDQPERSFGWKDQRKLWVGYQYVITGRNRSLEGRWEVAFKGSSEVFLPPDPIFASAMSENDNVFCAQLQCFHFPTLRHHDLHSWHAESCYEKSSFLCKRSQTCVDIKDNVVDEGFYFTPKGDDPCLSCTCHGGEPEMCVAALCERPQGCQQYRKDPKECCKFMCLDPDGNSLFDSMASGMRLVVSCISSFLILSLLLFMVHRLRQRRRERIESLIGANLHHFNLGRRIPGFDYGPDGFGTGLTPLHLSDDGEGGTFHFHDPPPPYTAYKYPDIGQPDDPPPPYEASIHPDSVFYDPADDDAFEPVEVSLPAPGDGGSEGALLRRLEQPLPTAGASLADLEDSADSSSALLVPPDPAQSGSTPAAEALPGGGRHSRSSLNTVV

SEQ ID NO: 8 - protein, 1859 aa, Artificial Sequence
  Synthetic
MVDKNIYIIQGEINIVVGAIKRNARWSTHTPLDEERDPLLHSFGHLKEVLNSITELSEIEPNVFLRPFLEVIRSEDTTGPITGLALTSVNKFLSYALIDPTHEGTAEGMENMADAVTHARFVGTDPASDEVVLMKILQVLRTLLLTPVGAHLTNESVCEIMQSCFRICFEMRLSELLRKSAEHTLVDMVQLLFTRLPQFKEEPKNYVGTNMKKLKMRAGGMSDSSKWKKQKRSPRPPRHMTKVTPGSELPTPNGTTLSSNLTGGMPFIDVPTPISSASSEAASAVVSPSTDSGLEFSSQTTSKEDLTDLEQPGSPGYSTATEPGSSELGVPEQPDLQEGTHVEKSQSASVESIPEVLEECTSPADHSDSASVHDMDYVNPRGVRFTQSSQKEGTALVPYGLPCIRELFRFLISLTNPHDRHNSEVMIHMGLHLLTVALESAPVAQCQTLLGLIKDEMCRHLFQLLSIERLNLYAASLRVCFLLFESMREHLKFQMEMYIKKLMEIITVENPKMPYEMKEMALEAIVQLWRIPSFVTELYINYDCDYYCSNLFEELTKLLSKNAFPVSGQLYTTHLLSLDALLTVIDSTEAHCQAKVLNSLTQQEKKETARPSCEIVDGTREASNTERTASDGKAVGMASDIPGLHLPGGGRLPPEHGKSGCSDLEEAVDSGADKKFARKPPRFSCLLPDPRELIEIKNKKKLLITGTEQFNQKPKKGIQFLQEKGLLTIPMDNTEVAQWLRENPRLDKKMIGEFVSDRKNIDLLESFVSTFSFQGLRLDEALRLYLEAFRLPGEAPVIQRLLEAFTERWMNCNGSPFANSDACFSLAYAVIMLNTDQHNHNVRKQNAPMTLEEFRKNLKGVNGGKDFEQDILEDMYHAIKNEEIVMPEEQTGLVRENYVWNVLLHRGATPEGIFLRVPTASYDLDLFTMTWGPTIAALSYVFDKSLEETIIQKAISGFRKCAMISAHYGLSDVFDNLIISLCKFTALSSESIENLPSVFGSNPKAHIAAKTVFHLAHRHGDILREGWKNIMEAMLQLFRAQLLPKAMIEVEDFVDPNGKISLQREETPSNRGESTVLSFVSWLTLSGPEQSSVRGPSTENQEAKRVALECIKQCDPEKMITESKFLQLESLQELMKALVSVTPDEETYDEEDAAFCLEMLLRIVLENRDRVGCVWQTVRDHLYHLCVQAQDFCFLVERAVVGLLRLAIRLLRREEISAQVLLSLRILLLMKPSVLSRVSHQVAYGLHELLKTNAANIHSGDDWATLFTLLECIGSGVKPPAALQATARADAPDAGAQSDSELPSYHQNDVSLDRGYTSDSEVYTDHGRPGKIHRSATDADVVNSGWLVVGKDDVDNSKPGPSRPGPSPLINQYSLTVGLDLGPHDTKSLLKCVESLSFIVRDAAHITPDNFELCVKTLRIFVEASLNGGCKSQEKRGKSHKYDSKGNRFKKKSKEGSMLRRPRTSSQHASRGGQSDDDEDEGVPASYHTVSLQVSQDLLDLMHTLHTRAASIYSSWAEEQRHLETGGQKIEADSRTLWAHCWCPLLQGIACLCCDARRQVRMQALTYLQRALLVHDLQKLDALEWESCFNKVLFPLLTKLLENISPADVGGMEETRMRASTLLSKVFLQHLSPLLSLSTFAALWLTILDFMDKYMHAGSSDLLSEAIPESLKNMLLVMDTAEIFHSADARGGGPSALWEITWERIDCFLPHLRDELFKQTVIQDPMPMEPQGQKPLASAHLTSAAGDTRTPGHPPPPEIPSELGACDFEKPESPRAASSSSPGSPVASSPSRLSPTPDGPPPLAQPPLILQPLASPLQVGVPPMTLPIILNPALIEATSPVPLLATPRPTDPIPTSEVN

SEQ ID NO: 9 - protein, 502 aa, Artificial Sequence [sequence omitted]

SEQ ID NO: 10 - protein, 397 aa, Artificial Sequence
  Synthetic
MDSLATSINQFALELSKKLAESAQGKNIFFSSWSISTSLTIVYLGAKGTTAAQMAQVLQFNRDQGVKCDPESEKKRKMEFNLSNSEEIHSDFQTLISEILKPNDDYLLKTANAIYGEKTYAFHNKYLEDMKTYFGAEPQPVNFVEASDQIRKDINSWVERQTEGKIQNLLPDDSVDSTTRMILVNALYFKGIWEHQFLVQNTTEKPFRINETTSKPVQMMFMKKKLHIFHIEKPKAVGLQLYYKSRDLSLLILLPEDINGLEQLEKAITYEKLNEWTSADMMELYEVQLHLPKFKLEDSYDLKSTLSSMGMSDAFSQSKADFSGMSSARNLFLSNVFHKAFVEINEQGTEAAAGSGSEIDIRIRVPSIEFNANHPFLFFIRHNKTNTILFYGRLCSP

SEQ ID NO: 11 - protein, 66 aa, Artificial Sequence
  Synthetic
MTGLSMDGGGSPKGDVDPFYYDYETVRNGGLIFAGLAFIVGLLILLSRRFRCGGNKKRRQINEDEP

SEQ ID NO: 12 - protein, 249 aa, Artificial Sequence
  Synthetic
MASRRMETKPVITCLKTLLIIYSFVFWITGVILLAVGVWGKLTLGTYISLIAENSTNAPYVLIGTGTTIVVFGLFGCFATCRGSPWMLKLYAMFLSLVFLAELVAGISGFVFRHEIKDTFLRTYTDAMQTYNGNDERSRAVDHVQRSLSCCGVQNYTNWSTSPYFLEHGIPPSCCMNETDCNPQDLHNLTVAATKVNQKGCYDLVTSFMETNMGIIAGVAFGIAFSQLIGMLLACCLSRFITANQYEMV

SEQ ID NO: 13 - protein, 321 aa, Artificial Sequence
  Synthetic
MGEWTILERLLEAAVQQHSTMIGRILLTVVVIFRILIVAIVGETVYDDEQTMFVCNTLQPGCNQACYDRAFPISHIRYWVFQIIMVCTPSLCFITYSVHQSAKQRERRYSTVFLALDRDPPESIGGPGGTGGGGSGGGKREDKKLQNAIVNGVLQNTENTSKETEPDCLEVKELTPHPSGLRTASKSKLRRQEGISRFYIIQVVFRNALEIGFLVGQYFLYGFSVPGLYECNRYPCIKEVECYVSRPTEKTVFLVFMFAVSGICVVLNLAELNHLGWRKIKLAVRGAQAKRKSIYEIRNKDLPRVSVPNFGRTQSSDSAYV

SEQ ID NO: 14 - protein, 514 aa, Artificial Sequence
  Synthetic
MALSELALVRWLQESRRSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIAPNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 15 - protein, 393 aa, Artificial Sequence
  Synthetic
METTMGFMDDNATNTSTSFLSVLNPHGAHATSFPFNFSYSDYDMPLDEDEDVTNSRTFFAAKIVIGMALVGIMLVCGIGNFIFIAALVRYKKLRNLTNLLIANLAISDFLVAIVCCPFEMDYYVVRQLSWEHGHVLCTSVNYLRTVSLYVSTNALLAIAIDRYLAIVHPLRPRMKCQTATGLIALVWTVSILIAIPSAYFTTETVLVIVKSQEKIFCGQIWPVDQQLYYKSYFLFIFGIEFVGPVVTMTLCYARISRELWFKAVPGFQTEQIRKRLRCRRKTVLVLMCILTAYVLCWAPFYGFTIVRDFFPTVFVKEKHYLTAFYIVECIAMSNSMINTLCFVTVKNDTVKYFKKIMLLHWKASYNGGKSSADLDLKTIGMPATEEVDCIRLK

SEQ ID NO: 16 - protein, 1212 aa, Artificial Sequence
  Synthetic
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCNSSLTLKTHHVQDSKMGFVINAIYSMAYGLHNMQMSLCPGYAGLCDAMKPIDGRKLLESLMKTNFTGVSGDTILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPKGSMGNGGRATMSSSNGKSVTWAQNEKSSRGQHLWQRLSIHINKKENPNQTAVIKPFPKSTESRGLGAGAGAGGSAGGVGATGGAGCAGAGPGGPESPDAGPKALYDVAEAEEHFPAPARPRSPSPISTLSHRAGSASRTDDDVPSLHSEPVARSSSSQGSLMEQISSVVTRFTANISELNSMMLSTAAPSPGVGAPLCSSYLIPKEIQLPTTMTTFAEIQPLPAIEVTGGAQPAAGAQAAGDAARESPAAGPEAAAAKPDLEELVALTPPSPFRDSVDSGSTTPNSPVSESALCIPSSPKYDTLIIRDYTQSSSSL

SEQ ID NO: 17 - protein, 381 aa, Artificial Sequence
  Synthetic
MGPIGAEADENQTVEEMKVEQYGPQTTPRGELVPDPEPELIDSTKLIEVQVVLILAYCSIILLGVIGNSLVIHVVIKFKSMRTVTNFFIANLAVADLLVNTLCLPFTLTYTLMGEWKMGPVLCHLVPYAQGLAVQVSTITLTVIALDRHRCIVYHLESKISKRISFLIIGLAWGISALLASPLAIFREYSLIEIIPDFEIVACTEKWPGEEKSIYGTVYSLSSLLILYVLPLGIISFSYTRIWSKLKNHVSPGAANDHYHQRRQKTTKMLVCVVVVFAVSWLPLHAFQLAVDIDSQVLDLKEYKLIFTVFHIIAMCSTFANPLLYGWMNSNYRKAFLSAFRCEQRLDAIHSEVSVTFKAKKNLEVRKNSGPNDSFTEATNV

SEQ ID NO: 18 - protein, 463 aa, Artificial Sequence
  Synthetic
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 19 - protein, 222 aa, Artificial Sequence
  Synthetic
MLWLLFFLVTAIHAELCQPGAENAFKVRLSIRTALGDKAYAWDTNEEYLFKAMVAFSMRKVPNREATEISHVLLCNVTQRVSFWFVVTDPSKNHTLPAVEVQSAIRMNKNRINNAFFLNDQTLEFLKIPSTLAPPMDPSVPIWIIIFGVIFCIIIVAIALLILSGIWQRRRKNKEPSEVDDAEDKCENMITIENGIPSDPLDMKGGHINDAFMTEDERLTPL

SEQ ID NO: 20 - protein, 6 aa, Artificial Sequence
  Synthetic
RRASAP

SEQ ID NO: 21 - protein, 7 aa, Artificial Sequence
  Synthetic
LRRASAP

SEQ ID NO: 22 - protein, 8 aa, Artificial Sequence
  Synthetic
WLRRASAP

SEQ ID NO: 23 - protein, 6 aa, Artificial Sequence
  Synthetic
RRATAP

SEQ ID NO: 24 - protein, 7 aa, Artificial Sequence
  Synthetic
LRRATAP

SEQ ID NO: 25 - protein, 8 aa, Artificial Sequence
  Synthetic
WLRRATAP

SEQ ID NO: 26 - protein, 6 aa, Artificial Sequence
  Synthetic
RRAYAP

SEQ ID NO: 27 - protein, 7 aa, Artificial Sequence
  Synthetic
LRRAYAP

SEQ ID NO: 28 - protein, 8 aa, Artificial Sequence
  Synthetic
WLRRAYAP

SEQ ID NO: 29 - protein, 6 aa, Artificial Sequence
  Synthetic
RRADAP

SEQ ID NO: 30 - protein, 7 aa, Artificial Sequence
  Synthetic
LRRADAP

SEQ ID NO: 31 - protein, 8 aa, Artificial Sequence
  Synthetic
WLRRADAP

SEQ ID NO: 32 - protein, 6 aa, Artificial Sequence
  Synthetic
RRAEAP

SEQ ID NO: 33 - protein, 7 aa, Artificial Sequence
  Synthetic
LRRAEAP

SEQ ID NO: 34 - protein, 8 aa, Artificial Sequence
  Synthetic
WLRRAEAP

SEQ ID NO: 35 - protein, 12 aa, Artificial Sequence
  Synthetic
RRASAPRRASAP

SEQ ID NO: 36 - protein, 14 aa, Artificial Sequence
  Synthetic
LRRASAPLRRASAP

SEQ ID NO: 37 - protein, 16 aa, Artificial Sequence
  Synthetic
WLRRASAPWLRRASAP

SEQ ID NO: 38 - protein, 12 aa, Artificial Sequence
  Synthetic
RRATAPRRATAP

SEQ ID NO: 39 - protein, 14 aa, Artificial Sequence
  Synthetic
LRRATAPLRRATAP

SEQ ID NO: 40 - protein, 16 aa, Artificial Sequence
  Synthetic
WLRRATAPWLRRATAP

SEQ ID NO: 41 - protein, 12 aa, Artificial Sequence
  Synthetic
RRAYAPRRAYAP

SEQ ID NO: 42 - protein, 14 aa, Artificial Sequence
  Synthetic
LRRAYAPLRRAYAP

SEQ ID NO: 43 - protein, 16 aa, Artificial Sequence
  Synthetic
WLRRAYAPWLRRAYAP

SEQ ID NO: 44 - protein, 12 aa, Artificial Sequence
  Synthetic
RRADAPRRADAP

SEQ ID NO: 45 - protein, 14 aa, Artificial Sequence
  Synthetic
LRRADAPLRRADAP

SEQ ID NO: 46 - protein, 16 aa, Artificial Sequence
  Synthetic
WLRRADAPWLRRADAP

SEQ ID NO: 47 - protein, 12 aa, Artificial Sequence
  Synthetic
RRAEAPRRAEAP

SEQ ID NO: 48 - protein, 14 aa, Artificial Sequence
  Synthetic
LRRAEAPLRRAEAP

SEQ ID NO: 49 - protein, 16 aa, Artificial Sequence
  Synthetic
WLRRAEAPWLRRAEAP

SEQ ID NO: 50 - protein, 13 aa, Artificial Sequence
  Synthetic
GRKKRRQRRRPPQ

SEQ ID NO: 51 - protein, 12 aa, Artificial Sequence
  Synthetic
AYARAAARQARA

SEQ ID NO: 52 - protein, 34 aa, Artificial Sequence
  Synthetic
DAATATRGRSAASRPTERPRAPARSASRPRRPVE

SEQ ID NO: 53 - protein, 27 aa, Artificial Sequence
  Synthetic
GWTLNSAGYLLGLINLKALAALAKKIL

SEQ ID NO: 54 - protein, 11 aa, Artificial Sequence
  Synthetic
PLSSISRIGDP

SEQ ID NO: 55 - protein, 16 aa, Artificial Sequence
  Synthetic
AAVALLPAVLLALLAP

SEQ ID NO: 56 - protein, 12 aa, Artificial Sequence
  Synthetic
AAVLLPVLLAAP

SEQ ID NO: 57 - protein, 15 aa, Artificial Sequence
  Synthetic
VTVLALGALAGVGVG

SEQ ID NO: 58 - protein, 21 aa, Artificial Sequence
  Synthetic
GALFLGWLGAAGSTMGAWSQP

SEQ ID NO: 59 - protein, 27 aa, Artificial Sequence
  Synthetic
GWTLNSAGYLLGLINLKALAALAKKIL

SEQ ID NO: 60 - protein, 18 aa, Artificial Sequence
  Synthetic
KLALKLALKALKAALKLA

SEQ ID NO: 61 - protein, 21 aa, Artificial Sequence
  Synthetic
KETWWETWWTEWSQPKKKRKV

SEQ ID NO: 62 - protein, 16 aa, Artificial Sequence
  Synthetic
KAFAKLAARLYRKAGC

SEQ ID NO: 63 - protein, 16 aa, Artificial Sequence
  Synthetic
KAFAKLAARLYRAAGC

SEQ ID NO: 64 - protein, 16 aa, Artificial Sequence
  Synthetic
AAFAKLAARLYRKAGC

SEQ ID NO: 65 - protein, 16 aa, Artificial Sequence
  Synthetic
KAFAALAARLYRKAGC

SEQ ID NO: 66 - protein, 16 aa, Artificial Sequence
  Synthetic
KAFAKLAAQLYRKAGC

SEQ ID NO: 67 - protein, 16 aa, Artificial Sequence
  Synthetic
AGGGGYGRKKRRQRRR

SEQ ID NO: 68 - protein, 11 aa, Artificial Sequence
  Synthetic
YGRKKRRQRRR

SEQ ID NO: 69 - protein, 11 aa, Artificial Sequence
  Synthetic
YARAAARQARA

SEQ ID NO: 70 - DNA, 20 bp, Artificial Sequence
  Synthetic
ggaggggtgaagacatgaga

SEQ ID NO: 71 - DNA, 22 bp, Artificial Sequence
  Synthetic
tctgaagtgattgtccatccag

SEQ ID NO: 72 - protein, 64 aa, Artificial Sequence
  Synthetic
MDRWYLGGSPKGDVDPFYYDYETVRNGGLIFAGLAFIVGLLILLSRRFRCGGNKKRRQINEDEP

SEQ ID NO: 73 - protein, 37 aa, Artificial Sequence
  Synthetic
HDEFERHAEGTFTSDVSSYLEGQAAKEFIAWLVKGRG

SEQ ID NO: 74 - protein, 33 aa, Artificial Sequence
  Synthetic
HADGSFSDEMNTILDNLAARDFINWLIQTKITD

SEQ ID NO: 75 - protein, 99 aa, Artificial Sequence
  Synthetic
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 76 - DNA, 533 bp, Artificial Sequence
  Synthetic
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatg